SEQUENCE LISTING (14 sequences)

SEQ ID NO: 1 - DNA, 7041 bp, Artificial Sequence
  Description of Artificial Sequence Synthetic polynucleotide
gatccggcagccggcggagcgctgctttcttggcaagcggtcgccagccccaacgccagggctgccagcccgaaacagcggggcaaggcagcttggaagggcgatcgcagcacgggcatggcaatgtctctctgaaggaatgcagaccttattcgtacagccagggttgaatcgtgggggtccaatcacttagctctgctgggctaaaccagagagcaatttcctgttgtgctgtttcgattgcatccgagccatggatgatgttgcggccaatattgacaccaaaatcaccacggatggtgcccggttctgccgtcagcggattggtagcgccgatcaacttgcgagcagccgccacaacgccttcgccttccaagacgatcgccacgatcggcccagaggtgatgaactcgacgaggccattgaagaaggggcgctcgcggtggacagcatagtgctgttcggccagctcgcgactgggcttcagctgctttaggcccaccagtttgaagcctttttgctcaaagcggccgatgatcgtaccgaccaaaccccgctgaacgccatcgggcttgatggcaataaatgtgcgttccacagacatctagatagtcctcaagacgaggcaagcattgagcttgccttcctatggttcgggatcactgggattcttgacaagcgatcgcggtcacatcgctatctcttaggacttcgcagcgggcgagtcggattgacccggtagggatttcgccagatcaatgcccgtggtttgtttcagcttctccagcaagctagcgatttgggtagcgctgccttccccttcgccaatcacagtgatcgactccacgtcgatatctggcacggtgcctgaaagcgtgacgagcagggactcgaagcttgcatgcctgcaggtcgactctagagctttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcataaagctctagaggatcggcggccgcaagctttctagattagtggtggtggtggtggtggctgctgccgcaggcatcgctgctaaaggcggccaggttggcatcttgcagggtccagcgtttaaagctatcaaacacggcgtagccttcgatggcaaacaggcggcctttcaggctatctttatcgggaaagatgcggctgctgatgctgtacaggccctgggtctgatcatcgcccacaaagatttcgatgctgctgcgatccagaaagatgtgcaggcgcactttgctggtgttgggcagggggcagctgcggatgcctttcacgttctggccgctgcggttgcgatccaggatgatgcggttctggctgcgatcgatgtacagcagggtttcctgacccttaccgttgcagcgcagggccaggccagcctggtaagcgctgctcttttccagatcaaagatcagttcgatttcttgcagggggctatccatggtaaaggggtgttcggcatcgctcagggtcaccacgctttcgatcttttcgctctggcgcaggatttccatttcgcgcacgggggtcatcacgattttgttatcgatcagatccagtttgcggggcagggtcatgcagccggcccagccatcgcgctggctgggtttctggttttcccacatatcaaaccaggcgatcaggatctggcggccatctttggcttcaaagcgctgggcggcgtaaaaatcgtggccgtaatccagttcctgaaagctggtttcgggggtaaactggggggcctgccatttgcccaggatgtagccgttctgaaacaggttgcggtttttgtagccgctggctttcaggccctgggggctaaacatcagcacgctgcggttgcccaggctaaagaaatcggggcattcccacataaaggcgcgtttgcccaggggcagctggctgttatcgcccagcagggttttcacaaagatccaatctttcaggttttcgctgcggtacagggccacgtggccgatgccctggtgtttttcatcatcggtgcggtagcccaccaccataaaccagtggttgttttctttccacacgcggggatcgcgaaagtgggccacctggggcatgggggctttttccagcacgatgccttctttctgaaagtggatgccatcgatgctggtcgccatgcactgcacttcgcggatggcatccaggctatcgttgctcagcacgatgtggccggtgtagatcagggtcagcacgccgttgttatccacggcgcagccgctaaagcagccatcgcgatcaaagctatcgccgggggccagggccactggcagggtttcccagtgcaccagatcgcggcttttagcgtgaccccagtgcattggaccccacactggagcaaaggggtagtactggtaaaacaggtggtattcgcctttgaaaaagatcaggccgttgggatcgttcatccagccggtcaggggggtcacgtgaaagccggggtaccattcgctgctcagcaggcgtttgccggctttcttctgggcatcttcggctttgatcaggtttttgtagctggggctttcactagacatttttttttcctccttgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgatgataagctgtcaaaccagatcaattcgcgctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgatctgctgcctcgcgcgtttcggtgatgacggtgaaaacctctgacacatgcagctcccggagacggtcacagcttgtctgtaactagtgggccccctagggaattctcgaggcctacttgtggatccccgggtaccgagctcgaatttcgagcttctggagcaggaagatgtcgcgggcattagcaccagcggtctgccaagcctccgccagccgttgggtcccttccgcttgagcttttccatcttcgacgatacgggcggcggccccccgcgcttccgcgatcgcccgtttacaagctgcctcagctggggcgatcacatcggcttgaagttgctgctgcacctgtttgatccgctcctgctgcacagggagttctgcttggctacgagcgacttcggtagcaatgtccgcttcagcttcggccaccaccgcttcgcgccgcgtcaacgcatcctgaatccggcgctcggcctcggcttgggcgatcgctacatcgcgatcgatccgacgcagggccgtgatcttgtcattttcggccgtttggatcgcagaggcagcctgggcatcggcttcagcaattcgggcatctcgctgcagatcagcccgctgcttgcgtccactagccgagagataaccgacctcatcggaaatgttctggacttgcagcgtatcgaggactagacccagctgctcaaggtcatcctccgcctcttccagcagacttttggcaaaggcaattttgtcctcgttgatctgctccggcgtgaggctggctaaaacaccacgcaagttgccttcgagggtctccttggcaatttgctcgatttccttacggtttttgccaagcagccgctcgatcgcgttgtggatggtcggttcttccccagcaatcttgatattggcaacgccttcaacagtcaggggaatgccgcccttggagaaggcattggaaacgcgcaactcaatgatcatgttggtcagatccat

SEQ ID NO: 2 - protein, 523 aa, Artificial Sequence
  Description of Artificial Sequence Synthetic polypeptide
MSSESPSYKNLIKAEDAQKKAGKRLLSSEWYPGFHVTPLTGWMNDPNGLIFFKGEYHLFYQYYPFAPVWGPMHWGHAKSRDLVHWETLPVALAPGDSFDRDGCFSGCAVDNNGVLTLIYTGHIVLSNDSLDAIREVQCMATSIDGIHFQKEGIVLEKAPMPQVAHFRDPRVWKENNHWFMVVGYRTDDEKHQGIGHVALYRSENLKDWIFVKTLLGDNSQLPLGKRAFMWECPDFFSLGNRSVLMFSPQGLKASGYKNRNLFQNGYILGKWQAPQFTPETSFQELDYGHDFYAAQRFEAKDGRQILIAWFDMWENQKPSQRDGWAGCMTLPRKLDLIDNKIVMTPVREMEILRQSEKIESVVTLSDAEHPFTMDSPLQEIELIFDLEKSSAYQAGLALRCNGKGQETLLYIDRSQNRIILDRNRSGQNVKGIRSCPLPNTSKVRLHIFLDRSSIEIFVGDDQTQGLYSISSRIFPDKDSLKGRLFAIEGYAVFDSFKRWTLQDANLAAFSSDACGSSHHHHHH

SEQ ID NO: 3 - DNA, 7151 bp, Artificial Sequence
  Description of Artificial Sequence Synthetic polynucleotide
agcttgtcatctgccggatgaggcaaaaccctgcctacggcgcgattacatcgtcccagcgcgatcgctcttactgttgatggctcgtgcttaaaaacaatgcaaacttcaccgtttcagctggtgattttcgactgtgatggtgtgcttgttgatagcggaacgcatcactaatcgcgtctttgcagacatgctcaatgaactgggtctgttggtgactttggatgacatgtttgagcagtttgtgggtcattccatggctgactgtctcaaactaattgagcgacggttaggcaatcctccaccccctgactttgttcagcactatcaacgccgtacccgtatcgcgttagaaacgcatctacaagccgttcctggggttgaagaggctttggatgctcttgaattgccctactgtgttgcgtccagtggtgatcatcaaaagatgcgaaccacactgagcctgacgaagctctggccacgatttgagggacgaatcttcagcgtgactgaagtacctcgcggcaagccatttcccgatgtctttttgttggccgccgatcgcttcggggttaatcctacggcctgcgctgtgatcgaagacacccccttgggagtagcggcaggcgtggcggcaggaatgcaagtgtttggctacgcgggttccatgcccgcttggcgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgggtaaccgatatctgtacaccgcggggtaccactagttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaaggaggaaaaaaaaatgtctagtgaaagcagccagggcctggtgacccgcctggccctgatcgctgctatcggtggtctgctgtttggctacgatagcgccgtgatcgccgccatcggtaccccagtggatatccactttatcgctccccgccacctgagcgctaccgccgccgccagcctgagcggtatggtggtggtggctgtgctggtcggctgcgtgaccggcagcctgctgagcggttggatcggcatccgctttggccgccgcggtggtctgctgatgagcagcatctgctttgtggccgccggctttggtgctgccctgaccgaaaaactgtttggtaccggtggtagcgctctgcaaatcttttgctttttccgctttctggccggcctgggcatcggcgtcgtgagcaccctgacccccacctacatcgccgaaatcgccccccccgataaacgcggtcagatggtcagcggccagcagatggctatcgtgaccggtgccctgaccggttacatctttacctggctgctggcccactttggcagcatcgattgggtgaacgccagcggttggtgctggagcccagccagcgaaggtctgatcggtatcgcctttctgctgctgctgctgaccgctccagataccccccactggctggtgatgaaaggccgccacagcgaagccagcaaaatcctggcccgcctggaaccccaggccgatcccaacctgaccatccagaaaatcaaagccggctttgataaagccatggataaaagcagcgccggcctgtttgcctttggcatcaccgtggtgtttgccggcgtgagcgtggccgcctttcagcagctggtcggcatcaacgccgtgctgtactacgccccccagatgtttcagaacctgggctttggcgccgataccgccctgctgcaaaccatcagcatcggcgtggtgaactttatctttaccatgatcgccagccgcgtggtggatcgctttggtcgcaaaccactgctgatctggggtgccctgggcatggctgctatgatggctgtgctgggctgctgcttttggtttaaagtgggcggcgtgctgcccctggccagcgtgctgctgtacatcgccgtgtttggcatgagctggggccccgtgtgctgggtggtgctgagcgaaatgtttcccagcagcatcaaaggcgccgccatgcccatcgccgtgaccggccagtggctggccaacatcctggtgaactttctgtttaaagtggccgatggcagccccgccctgaaccagacctttaaccacggctttagctacctggtgtttgccgccctgagcatcctgggcggcctgatcgtggcccgctttgtgcccgaaaccaaaggccgcagcctggatgaaatcgaagaaatgtggcgcagccagaaaggcagcagccaccaccaccaccaccactaatctagaaagcttgcggccgcgctagcaagcttggccggatccggccggatccgggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttattgagccgttaccccacctactagctaatcccatctgggcacatccgatggcaagaggcccgaaggtccccctctttggtcttgcgacgttatgcggtattagctaccgtttccagtagttatccccctccatcaggcagtttcccagacattactcacccgtccgccactcgtcagcaaagaagcaagcttagatcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgatctggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatcc

SEQ ID NO: 4 - protein, 482 aa, Artificial Sequence
  Description of Artificial Sequence Synthetic polypeptide
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIAPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQKGSSHHHHHH

SEQ ID NO: 5 - DNA, 1044 bp, Artificial Sequence
  Description of Artificial Sequence Synthetic polynucleotide
aaggaggaaaaaaaaatgtctagacatcaccatcatcatcaccctaggaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaagcggccgcg

SEQ ID NO: 6 - protein, 338 aa, Artificial Sequence
  Description of Artificial Sequence Synthetic polypeptide
MSRHHHHHPRKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 7 - DNA, 1718 bp, Artificial Sequence
  Description of Artificial Sequence Synthetic polynucleotide
ggaggaaaaaaaaatgtctagaaagcttgcggccgcacatcaccatcatcatcacaatctctggcaacaaaactacgatcccgccgggaatatctggctttccagtctgatagcatcgcttcccatcctgtttttcttctttgcgctgattaagctcaaactgaaaggatacgtcgccgcctcgtggacggtggcaatcgcccttgccgtggctttgctgttctataaaatgccggtcgctaacgcgctggcctcggtggtttatggtttcttctacgggttgtggcccatcgcgtggatcattattgcagcggtgttcgtctataagatctcggtgaaaaccgggcagtttgacatcattcgctcgtctattctttcgataacccctgaccagcgtctgcaaatgctgatcgtcggtttctgtttcggcgcgttccttgaaggagccgcaggctttggcgcaccggtagcaattaccgccgcattgctggtcggcctgggttttaaaccgctgtacgccgccgggctgtgcctgattgttaacaccgcgccagtggcatttggtgcgatgggcattccaatcctggttgccggacaggtaacaggtatcgacagctttgagattggtcagatggtggggcggcagctaccgtttatgaccattatcgtgctgttctggatcatggcgattatggacggctggcgcggtatcaaagagacgtggcctgcggtcgtggttgcgggcggctcgtttgccatcgctcagtaccttagctctaacttcattgggccggagctgccggacattatctcttcgctggtatcactgctctgcctgacgctgttcctcaaacgctggcagccagtgcgtgtattccgttttggtgatttgggggcgtcacaggttgatatgacgctggcccacaccggttacactgcgggtcaggtgttacgtgcctggacaccgttcctgttcctgacagctaccgtaacactgtggagtatcccgccgtttaaagccctgttcgcatcgggtggcgcgctgtatgagtgggtgatcaatattccggtgccgtacctcgataaactggttgcccgtatgccgccagtggtcagcgaggctacagcctatgccgccgtgtttaagtttgactggttctctgccaccggcaccgccattctgtttgctgcactgctctcgattgtctggctgaagatgaaaccgtctgacgctatcagcaccttcggcagcacgctgaaagaactggctctgcccatctactccatcggtatggtgctggcattcgcctttatttcgaactattccggactgtcatcaacactggcgctggcactggcgcacaccggtcatgcattcaccttcttctcgccgttcctcggctggctgggggtattcctgaccgggtcggatacctcatctaacgccctgttcgccgcgctgcaagccaccgcagcacaacaaattggcgtctctgatctgttgctggttgccgccaataccaccggtggcgtcaccggtaagatgatctccccgcaatctatcgctatcgcctgtgcggcggtaggcctggtgggcaaagagtctgatttgttccgctttactgtcaaacacagcctgatcttcacctgtatagtgggcgtgatcaccacgcttcaggcttatgtcttaacgtggatgattccttaagcggccgccg

SEQ ID NO: 8 - protein, 564 aa, Artificial Sequence
  Description of Artificial Sequence Synthetic polypeptide
MSRKLAAAHHHHHHNLWQQNYDPAGNIWLSSLIASLPILFFFFALIKLKLKGYVAASWTVAIALAVALLFYKMPVANALASVVYGFFYGLWPIAWIIIAAVFVYKISVKTGQFDIIRSSILSITPDQRLQMLIVGFCFGAFLEGAAGFGAPVAITAALLVGLGFKPLYAAGLCLIVNTAPVAFGAMGIPILVAGQVTGIDSFEIGQMVGRQLPFMTIIVLFWIMAIMDGWRGIKETWPAVVVAGGSFAIAQYLSSNFIGPELPDIISSLVSLLCLTLFLKRWQPVRVFRFGDLGASQVDMTLAHTGYTAGQVLRAWTPFLFLTATVTLWSIPPFKALFASGGALYEWVINIPVPYLDKLVARMPPVVSEATAYAAVFKFDWFSATGTAILFAALLSIVWLKMKPSDAISTFGSTLKELALPIYSIGMVLAFAFISNYSGLSSTLALALAHTGHAFTFFSPFLGWLGVFLTGSDTSSNALFAALQATAAQQIGVSDLLLVAANTTGGVTGKMISPQSIAIACAAVGLVGKESDLFRFTVKHSLIFTCIVGVITTLQAYVLTWMIP

SEQ ID NO: 9 - DNA, 4885 bp, Artificial Sequence
  Description of Artificial Sequence Synthetic polynucleotide
ctagtcgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtctagttcagccagctcgtcgtgatgtcgaaacccaagccaccctcagaggtgaaggccgcttcgagcacatcgggaagcgtgtcgacgacggtgctcggtgcctcgggtaagagccagatagatgcggtgacgttcaccgtcgcgatcgtggcgggaaccacggtcactgcgtccgttacaactcgcacgtcgtcggccagcacgactgactcgacggcttcgattagcccgggagaggcaaggccatcaggctcggtagacagaatgctgattaacacctcgccgggagcagggctgctcacagccgcgtccttcacccgtgggtcagccgtcagcgcttgatagcggtaccaggccgcaccgcctgcggtcgagctgcccttgatccgctcgatggtgcgatcgcgaagctcgccatccggctcattcggctgccgcaccacgccgtagaacgcggcaaggttgtcgaggtcggggccgtttgagtagcgaagcagtgtcgcaagaagtgcgtcgttgatccgctgacgcaggatcagctcgcgggctgcgcagacctccagcagcttgatgaccgggtcggattcgaggatggcggtgtagctggcgtcacgcgatcgcaggtcgtcgatcaagtcctgcaggatcagttcaaagtccagcgcttcgatgatggtgggcgcgggaatcgtagcaaagtcaagaacggtcatgagacgactaagccctccagcgtgatacgcctgccctcggggatgtagtagccgatcaggttcagctcgacttgaccagctgcgctggctgagacgatgcgaaccttctccagcttcagccgtggctcccagcgatcgagcgcttcagctgtggccgccaccaggtcaacgatgagggactggttgatcggtctagtcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactctagtttcgaattgtgagcgctcacaatttcgaaaccccaggctttacactttatgcttccggctcgtataatgtgtggaattgtgagcggataacaatttcacacaaggaggaaaaacatatgtctagaaagcttgcggccgcgtaggatccgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttgaattcctactagtcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgatcccacagccgccagttccgctggcggcattttaactttctttaatgaatctagtgacaagccggggcagacgtgagccgtagtcccgtcgccagacgcgggtgcccacgggcgtcgtcaggatgtccgtaattgactgccggaggtggtcaatgcccttcagctccttgccactgtcacggctcatgcctcgggtcattagtcgcccgctccggtatcttcactggcttcgatgattgccgccccgcagctgcagaggtcaccgatccgagcagtcggcctctggttggtaaagaccgtgcgactgccggtgatgatcgtgttcaagccatgcagggggcaggcgtgaaggtcatcctttcgggccacggggcggctgttgacgaaggtgtcgtcgctcccggtgatgatgatcccgccgtgatcggtcacgtcgtttagtcgagcgatgcctggcgtcgtagtcacgggtttaggtcaatacgacttgcggtcactgtaacgttgccctcggcggtcacgttaacgtcgccttgggcttcgacttgcgcctcctgcacaaggatcacaatccgtccttgggctgcggtgaggtcgatcttgtactcatgcgcttcgcggtcgtactggatgattgagtcatcctcgaactgcgtcttttggatcgtttctttgtcctcgatctgggggtagtcagtcgagaacgcgccgggcatcgcgaagccctgactgatctcgccggagggggccatcacgacgacggcctcaccgacctcgggcgcccaccagaaccgatccttgcccgctcgctgcgtgagccacggaatccagtcagtgaggagcagcgcctcgccgctctcctcgtcttcctcgatcgcgacacggatcagccccttgggatagtcagcctcggctaccctgcctacgcggagcaagttgccgtgacgccgactgtctcgagtat

SEQ ID NO: 10 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatag

SEQ ID NO: 11 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 12 - DNA, 1206 bp, Escherichia coli
atgtttcatctcgatactttagcaacgcttgttgccgcaacgctgacgttgctgctcgggcgtaagttggtccattccgtctcctttttgaagaaatacaccataccggaacctgttgcgggtggtttgttggtggcgctggcgctactagtactgaaaaaaagcatgggctgggaagtcaactttgatatgtccctgcgcgatccgttaatgctggctttcttcgccaccattggcctgaacgccaacattgccagtttgcgtgccggtgggcgtgtggttggcatcttcttgattgtggttgttggtctgttggtgatgcaaaatgccattggcattggtatggctagcttgttagggcttgatccgctgatggggctgttggccggttctattacgctttccggcggtcacggtacgggcgctgcgtggagtaaattgttcattgaacgttatggcttcaccaatgcgacggaagtggcgatggcctgtgcaacgttcggtctggtgctgggcggcttgattggcggtccggtggcgcgctatctggtgaaacactccaccacgccgaacggtattccggatgaccaggaagtcccgacggcgtttgaaaagccggatgtgggacgcatgatcacctcgttggtgctgattgaaactatcgcgctgattgctatctgcctgacggtggggaaaattgttgcgcaacttttggctggcactgcttttgaactgccgaccttcgtctgtgtactgtttgttggcgtgattctgagcaacggtctgtcaataatgggcttttaccgcgtctttgagcgtgcggtatccgtgctgggtaacgtaagcttgtcgttgttcctggcgatggcgctgatggggctgaaactgtgggagctggcttcgctggcgctgccgatgctggcgattctggtggtacagaccatcttcatggcgttgtatgccatcttcgttacctggcgcatgatgggcaaaaactacgatgcggcagtgctggctgcgggtcactgtggttttggcctcggtgcaacgccaacggcaatcgccaacatgcaggcgatcactgaacgctttggcccgtcgcacatggcgtttttggtggtgccgatggtcggtgcgttctttatcgatatcgtcaatgcgctggtgattaagctgtatttaatgttgccgatttttgccggttaa

SEQ ID NO: 13 - protein, 401 aa, Escherichia coli
MFHLDTLATLVAATLTLLLGRKLVHSVSFLKKYTIPEPVAGGLLVALALLVLKKSMGWEVNFDMSLRDPLMLAFFATIGLNANIASLRAGGRVVGIFLIVVVGLLVMQNAIGIGMASLLGLDPLMGLLAGSITLSGGHGTGAAWSKLFIERYGFTNATEVAMACATFGLVLGGLIGGPVARYLVKHSTTPNGIPDDQEVPTAFEKPDVGRMITSLVLIETIALIAICLTVGKIVAQLLAGTAFELPTFVCVLFVGVILSNGLSIMGFYRVFERAVSVLGNVSLSLFLAMALMGLKLWELASLALPMLAILVVQTIFMALYAIFVTWRMMGKNYDAAVLAAGHCGFGLGATPTAIANMQAITERFGPSHMAFLVVPMVGAFFIDIVNALVIKLYLMLPIFAG

SEQ ID NO: 14 - protein, 6 aa, Artificial Sequence
  Description of Artificial Sequence Synthetic 6xHis tag
HHHHHH

(19) United States
(12) Patent Application Publication
(10) Pub. No.: US 2012/0220007 A1
(43) Pub. Date: Aug. 30, 2012

(54) PRODUCTION OF SECRETED BIOPRODUCTS FROM PHOTOSYNTHETIC MICROBES

(75) Inventors: Jeffrey Charles Way, Cambridge, MA (US); Henrike Niederholtmeyer, Lausanne (CH); Bernd Wolfstaedter, Schwaigern (DE); David Savage, Cambridge, MA (US)

(73) Assignee: PRESIDENT AND FELLOWS OF HARVARD COLLEGE, Cambridge, MA (US)

(21) Appl. No.: 13/394,070

(22) PCT Filed: Sep. 3, 2010
(86) PCT No.: PCT/US2010/047830

§ 371 Date: May 11, 2012

Related U.S. Application Data
(60) Provisional application No. 61/239,985, filed on Sep. 4, 2009.
(60) Provisional application No. 61/317,001, filed on Mar. 24, 2010.

(51) Int. Cl. C12P 1/04; C12N 1/13

(52) U.S. Cl. 435/170; 435/257.2

(57) ABSTRACT

The invention provides new compositions and methods for the production of useful chemicals from recombinant photosynthetic bacteria, such as recombinant cyanobacteria. Embodiments of the invention provide for the genetic engineering of photosynthetic bacteria to express and secrete a product of interest, such as hexose sugars or lactate. More specifically, the recombinant cyanobacteria express heterologous genes for both product expression and secretion. In some embodiments, the recombinant cyanobacteria express heterologous genes for secretion of products coupled to a pH gradient across a cell membrane. Sugars, such as sucrose, glucose and fructose, and other useful chemicals, such as lactic acid, are produced via the invention.

### CROSS-REFERENCE TO RELATED APPLICATIONS

This International application claims the benefit of priority under 35 U.S.C. §119(e) of U.S. Provisional Application Nos. 61/317,001, filed Mar. 24, 2010 and 61/239,985 filed Sep. 4, 2009, the contents of each of which are incorporated herein by reference in its entirety.

### FIELD OF THE INVENTION

The invention relates to compositions and methods for the production of useful chemicals from photosynthetic microbes.

### BACKGROUND

The cost of commodity chemicals limits the growth of the world economy. Currently chemicals, such as sugars, are made from green plants with limited geographical distribution, while other natural products such as lactic acid and succinic acid are made by metabolic engineering using sugars as a starting material. These carbon-based molecules are ultimately derived from carbon dioxide fixation, but the existing processes are inefficient in many ways. For example, in the production of sugar by green plants, most of the fixed carbon goes into the biomass of the plant, such as cellulosic biomass that is relatively resistant to further breakdown.

Ideally, photosynthetic microbes could directly produce such molecules, much as ethanol is produced by yeast, because of the efficient use of light energy by these organisms and the potential for CO₂ mitigation during production. Currently, most photosynthetic bacteria do not secrete carbon-based molecules, likely because of the metabolic cost of their synthesis. In addition, and unlike ethanol, many hydrophilic molecules such as glucose, lactic acid and succinic acid cannot pass through cell membranes under normal conditions. Therefore, there is a need in the art for methods and systems that allow the production and secretion of hydrophilic molecules from photosynthetic bacteria.

### SUMMARY

The present invention provides compositions and methods for the production of useful chemicals from photosynthetic bacteria, such as cyanobacteria.

In one embodiment, the invention provides genetically engineered photosynthetic cyanobacteria that express at least one heterologous enzyme and at least one heterologous transporter, wherein the transporter mediates the secretion of a product into an extracellular medium. For example, the transporter mediates the secretion of a product whose synthesis is enhanced by the heterologous enzyme. In a particular embodiment, the heterologous enzyme is an invertase, such as invA; and the transporter is glf. In another embodiment, the heterologous enzyme is a dehydrogenase, such as ldhA, and the transporter is lldP. The cyanobacteria may further include a resistance marker.

In some embodiments, the heterologous transporter is a member of the major facilitator superfamily, including the Glut family of glucose transport proteins, the HXT family of glucose transporters, lacY, lldP, a mammalian citric acid transport protein, the glpT glycerol phosphate transporter, a sucrose transporter, an amino acid transporter, a glutamate transporter, and GLF. In some embodiments, the heterologous hexose (e.g., glucose) transporters can be the Glut, HXT, and GLF proteins.

In further embodiments, the recombinant photosynthetic microbes further comprise additional heterologous genes that encode enzymes for enhancing synthesis of intracellular precursors. In a particular embodiment, the additional heterologous gene is udhA, which encodes NAD(P)H transhydrogenase. In specific embodiment, the additional heterologous gene is galU, which encodes UDP-glucose phosphorylase. In alternative embodiments, the additional heterologous gene encodes an enzyme for sucrose synthesis, such as sucrose phosphate synthase and sucrose phosphate phosphatase.

In yet further embodiments, the recombinant microbe further comprises at least one reduction-of-function mutation that can enhance production of hydrophilic molecules. In some embodiments, the reduction-of-function mutation is a deletion in at least one endogenous kinase gene that phosphorylates a hydrophilic product. In a particular embodiment, the endogenous kinase is hexokinase. In another embodiment, the endogenous kinase is glucokinase. In a particular embodiment, the reduction-of-function mutation is a deletion in genes encoding any membrane-associated NAD(P) transhydrogenase.

In some embodiments, genetically engineered photosynthetic microbes are cyanobacteria. In some embodiments, the transporter is a member of the major facilitator superfamily. In some embodiments, the compound is a chiral compound. Examples of chiral compounds include, without limitation, sugars and carboxylic acids, such as lactic acid. Examples of sugars include glucose, fructose, a mixture of glucose and fructose, or sucrose.

In some embodiments, the enzyme is expressed from an inducible promoter. Non-limiting examples of inducible promoters include a lac operon promoter, a nitrogen-sensitive promoter, and a salt-inducible promoter. In one embodiment, the inducible promoter is IPTG-inducible promoter. In another embodiment, the inducible promoter is NaCl-inducible promoter.

In another aspect, the invention provides for a recombinant photosynthetic bacterium, such as cyanobacterium, that expresses at least one heterologous transporter that is coupled to the pH gradient across a cell membrane. In some embodiments, the invention provides for a recombinant photosynthetic bacterium, such as a cyanobacterium, that expresses at least one heterologous transporter that co-transports a proton with a product (e.g., a metabolite). In alternative embodiments, the invention provides genetically engineered photosynthetic bacteria, such as cyanobacteria, that express a heterologous transporter that co-transports a sodium ion with a product (e.g., a metabolite).

In some embodiments, the heterologous transporter mediates the symport or antiport of a product and an inorganic ion. Examples of an inorganic ion include a proton, a sodium ion, a potassium ion, a chloride ion, and a hydroxide ion.

In some embodiments, the heterologous transporter can be a lactic acid/proton symporter, a sucrose proton symporter, a lactose/proton symporter, a dicarboxylic acid/proton symporter, an amino acid/proton symporter, a citrate/proton symporter, an amino acid/sodium ion symporter, or an amino acid/hydroxide ion antiporter. Examples of such heterologous transporters include, but not limited to, a sucrose transporter CscB, a lactose permease of E. coli encoded by the lacY gene, a mammalian H⁺/peptide transporter PepT1H, a glutamate transporter GltS, and a lactate transporter lldP.

In yet a another aspect, the invention provides a method for producing a hydrophilic product, the method comprising culturing a recombinant cyanobacterium according to the invention in the presence of light and CO₂, and obtaining the product from a supernatant of the cultured microbe. In some aspects, the method further includes providing an inducing agent to induce the expression of at least one heterologous gene expressed in the recombinant bacterium. In one embodiment, the heterologous gene encodes an enzyme, a transporter or both. In particular embodiments, the hydrophilic product is glucose and/or fructose, or lactic acid.

In yet another aspect, the invention provides a method for producing a hydrophilic product, the method comprising culturing a cyanobacteria according to the second aspect of the invention in the presence of light and CO₂, and obtaining said product from a supernatant of the cultured microbe. In some aspects the method further includes providing an inducing agent to induce the expression of at least one heterologous gene expressed in cyanobacteria. In one embodiment, the heterologous gene encodes an enzyme, a transporter or both. In a particular embodiment, the hydrophilic product is lactic acid, sucrose, an amino acid, citric acid, malic acid, fumaric acid, or succinic acid.

### DESCRIPTION OF THE DRAWINGS

FIGS. 1A and 1B show schematic diagrams of engineering strategies for production of bioproducts by a genetically engineered cyanobacterium. FIG. 1A illustrates a cyanobacterial cell in which one or more heterologous enzymes have been expressed to produce a hydrophilic metabolite that cannot spontaneously cross a cell membrane, and in which a heterologous transporter has been introduced to allow secretion of the metabolite from the cell. FIG. 1B shows an engineering scheme for production of hexose sugars (e.g., glucose and fructose) by a genetically engineered Synechococcus elongatus 7942. Synechococcus naturally produces sucrose in response to salt stress by fixing carbon via natural pathways such as Calvin Cycle. The bacteria are engineered to express invertase (encoded by invA), which cleaves sucrose into glucose and fructose, and the glf gene encoding a glucose- and fructose-facilitated diffusion transporter, which allows export of the sugars from the cell. Color versions of drawings are available in Niederholtmeyer et al., 76 Appl. Environ. Microbio. 3462 (2010).

FIG. 2 shows plasmid vectors that mediate integration of the invA and glf genes into the S. elongatus 7942 genome by homologous recombination. Derivatives of S. elongatus were constructed by insertion of transgenes into “neutral sites” NS1 and NS2 with spectinomycin-resistance and kanamycin-resistance markers, respectively. Strains lacking a particular transgene were transformed with empty spectinomycin-resistant and kanamycin-resistant vectors as controls.

FIGS. 3A and 3B show the gene and protein sequences of the inserted invA into S. elongatus 7942. FIG. 3A shows the sequence of invA, spectinomycin-resistance, and lad genes inserted into NS1 of S. elongatus 7942. The region encoding the invA gene plus immediately flanking regions is shown in capital letters. This gene is encoded in the antisense orientation. The start and stop codons are underlined. The plasmid carrying the invA gene is termed DS1321. FIG. 3B shows the sequence of the invertase protein, including a His₆ tag at the C-terminus, as expressed in S. elongatus 7942.

FIGS. 4A and 4B show the gene and protein sequences of the inserted glf into S. elongates 7942. FIG. 4A shows the sequence of glf, kanamycin-resistance, and lad genes inserted into NS2 of S. elongatus 7942. The region encoding glf and immediately flanking sequences are shown in capitals, with the start and stop codons underlined. The plasmid carrying the glf gene is termed DS21. FIG. 4B shows the sequence of the GLF protein, including a His6 tag at the C-terminus, as expressed in S. elongatus 7942.

FIGS. 5A to 5D show the expression of transgenes to promote hexose sugar or lactate synthesis and secretion. FIG. 5A shows the western blot assay for expression of His₆-tagged invA and glf in E. coli and Synechococcus. Predicted molecular weights of tagged InvA and GLF are 60,000 and 55,400 respectively. Symbols ‘−’ and ‘+’ indicate samples growth without or with 1 mM IPTG. Lane 1—E. coli, invA expression plasmid; lane 2—E. coli, glf expression plasmid; lane 3—S. elongatus, no plasmid; lane 4—S. elongatus, invA expression plasmid; lane 5—S. elongatus, glf expression plasmid; lane 6—S. elongatus, invA and glf expression plasmids. (The glf product was not detected.) FIG. 5B shows a western blot assay for expression of His₆-tagged ldhA and lldP in Synechococcus containing the ldhA and lldP expression constructs. Lanes 1 to 3: 100 mM, 1 mM, and 0 IPTG, respectively. (The lldP product was not detected.) FIG. 5C shows invertase enzyme activity from extracts of Synechococcus expressing invA, glf, or both transgenes. FIG. 5D shows functional expression of the glf transporter in Synechococcus, assayed by growth of Synechococcus in the dark. Solid lines: growth; dotted lines: disappearance of glucose from culture medium. The lines that remain fairly constant correspond to wild-type S. elongatus 7942; the decreasing dotted line and increasing solid line correspond to recombinant S. elongatus 7942 that expresses the glf gene.

FIGS. 6A and 6B show sugar production in culture medium of Synechococcus strains expressing Z. mobilis glf and invA (cross), only glf (triangle), only invA (square), or neither transgene (diamond). The x axis shows the time after induction of sucrose synthesis with 200 mM NaCl and of transgenes with 100 μM IPTG. Symbol “21” refers to plasmid DS21; “13” refers to plasmid DS1321. FIG. 6A shows extracellular concentration of glucose of the four strains. FIG. 6B shows extracellular concentration of fructose of the four strains.

FIG. 7 shows the growth curves of cyanobacteria engineered to express invA and glf from Z. mobilis (square), only glf (cross), only invA (triangle), or neither transgene (diamond). The x axis shows the time after induction of sucrose synthesis with 200 mM NaCl and of transgenes with 100 μM IPTG.

FIG. 8 shows the extracellular concentration of sucrose produced by Synechococcus strains expressing Z. mobilis glf and invA (glf+invA), only glf (13+glf), only invA (21+invA), or neither transgene (21+13). The bars show the concentrations of both intracellular and extracellular sucrose determined from a Day-3 whole culture sample (cells and culture supernatant); less than 3 μM sucrose was detected in the invA-expressing strains.

FIGS. 9A and 9B show growth and sugar production or consumption as a function of a cycle consisting of 12-h days and 12-h nights. The lines indicate OD₇₅₀; bars indicate the concentrations of extracellular fructose (gray bars) and glucose (striped bars). The arrows indicate the induction with NaCl and IPTG at dawn (FIG. 9A) and at dusk (FIG. 9B).

FIGS. 10A to 10C show the growth and sugar production of glf+invA-expressing and control cells as a function of NaCl concentration. FIG. 10A shows the growth rates of a Synechococcus empty vector control. Symbol “21+13” and glf+invA strain after induction with 100 mM IPTG and various different NaCl concentrations in BG-11 medium. FIG. 10B shows the concentrations of glucose and fructose in the culture medium of a Synechococcus glf+invA strain four days after induction with 100 mM IPTG and various different NaCl concentrations in BG-11 medium. FIG. 10C shows the extracellular concentrations of glucose and fructose produced by a Synechococcus glf+invA strain on a per cell basis.

FIGS. 11A to 11E show that sugar-secreting Synechococcus supports E. coli growth in coculture. For FIGS. 11A to 11C, E. coli DH5α containing a YFP expression plasmid was diluted to obtain a concentration of 10⁶ cells/ml in wild-type Synechococcus cultures or Synechococcus cultures expressing glf and invA in BG-11 medium with 200 mM NaCl, 1 mg/ml NH₄Cl, and appropriate antibiotics. FIG. 11A shows continued growth of engineered and wild-type Synechococcus in the presence of E. coli. FIGS. 11B to 11C show growth of E. coli in the presence of engineered or wild-type Synechococcus strains, as determined by the number of CFU (FIG. 11B) or YFP fluorescence (FIG. 11C). FIGS. 11D to 11E show coculture of sugar-secreting Synechococcus and E. coli on agar plates at two different NaCl concentrations. E. coli YFP fluorescence (lighter shade); Synechococcus chlorophyll autofluorescence (coiled features). Microcolonies formed on BG-11 agar with 100 mM NaCl (FIG. 11D) and 200 mM NaCl (FIG. 11E) four days after plating.

FIGS. 12A to 12C show plasmid schematics for expression of ldhA and lldP in S. elongatus 7942. FIG. 12A shows the plasmid for expression of ldhA. FIG. 12B shows the plasmid for expression of lldP. FIG. 12C shows construction of derivatives of S. elongatus by insertion of lldP and ldhA genes into “neutral sites” NS1 and NS2 with spectinomycin-resistance and kanamycin-resistance markers, respectively. Strains lacking a particular transgene were transformed with empty spectinomycin-resistant and kanamycin-resistant vectors as controls.

FIGS. 13A to 13B show the sequence of the inserted ldhA gene and expressed protein in S. elongatus 7942. FIG. 13A shows the nucleotide sequence of the inserted ldhA gene in S. elongatus. Start and stop codons are underlined. FIG. 13B shows the amino acid sequence of ldhA gene encoding lactate dehydrogenase, including a His₆ tag at the N-terminus, as expressed in S. elongatus 7942.

FIGS. 14A and 14B show the sequence of the inserted lldP gene and expressed protein in S. elongatus 7942. FIG. 14A shows the nucleotide sequence of the inserted lldP gene in S. elongatus. Start and stop codons are underlined. FIG. 14B shows the amino acid sequence of the expressed protein encoded by lldP, including a His₆ tag at the N-terminus, as expressed in S. elongatus 7942.

FIGS. 15A to 15C show schematic diagrams for production of bioproducts by a recombinant cyanobacterium that expresses an ion-coupled transporter. FIG. 15A illustrates a cyanobacterial cell in which an ion-coupled transporter is expressed to allow secretion of the product from the cell in a manner that is assisted by the concentration gradient of the ion. FIG. 15B shows the engineering scheme for cyanobacterial production of lactic acid (lactate). Synechococcus naturally produces pyruvate as a metabolic intermediate by fixing carbon via Calvin cycle. The bacteria are engineered to express both lactate dehydrogenase (ldhA), which produces lactic acid, and a transporter (lldP), which cotransports lactate and H⁺ from the cell. FIG. 15C shows the engineering scheme for cyanobacterial production of sucrose. Synechococcus naturally produces intracellular sucrose in response to salt stress. The bacteria are engineered to express the cscB gene, which encodes a proton-coupled sucrose transporter.

FIGS. 16A and 16B show lactate production and growth of Synechococcus strains Synechococcus strains expressing E. coli ldhA and lldP (cross), only lldP (triangle), only ldhA (square), or neither transgene (diamond). FIG. 16A shows the concentration of secreted lactic acid in culture medium of these four strains. FIG. 16B shows the growth of these four strains.

FIGS. 17A to 17C show rational metabolic engineering of Synechococcus to enhance lactate production. FIG. 17A shows a schematic depiction of a model for transhydrogenase action. NADPH is the major carrier of reducing equivalents in photosynthetic microbes. Exchange with NAD is catalyzed by NADP/NAD transhydrogenase to yield NADH, the reducing agent substrate for pyruvate dehydrogenase. FIG. 17B shows lactate concentrations in culture supernatants of induced Synechococcus ldhA+lldP with or without an udhA expression construct. Dashed lines: lactate concentrations; solid lines: bacterial density. FIG. 17C shows the insertion of udhA into “neutral site” NS3 with a chloramphenicol-resistance marker.

FIGS. 18A and 18B depict a new vector, pHN1-LacUV5, for integration of transgenes into the S. elongatus 7942 genome. This vector allows integration at the “NS3” site, that corresponds to a sequence within the remnant of a cryptic prophage, such that integrations are expected to have no phenotypic consequences related to integration per se. FIG. 18A shows sites of NS3, transcription terminators, lac operon promoter and multiple cloning sites for genes to be expressed. The sequence of pHN1-LacUV5 vector in FIG. 18B includes the origin of replication from pUC57 (bases 6 to 625); an “NS3-II” segment from bases 655 to 1554; a segment containing the E. coli rrnB T1 and T2 terminators from bases 1567 to 1769; the lacUV5 promoter from bases 1791 to 1881; a multiple cloning site including unique NdeI, XbaI, HindIII, NotI and BamHI sites from bases 1900 to 1934; a coding sequence of a chloramphenicol resistance gene from bases 2037 to 2696; an E. coli lad coding sequence from bases 2839 to 3921; an E. coli Trp operon terminator from bases 3922 to 3972; and an “NS3-I” segment from bases 3977 to 4876. Analogous plasmids that use the weaker wild-type (not UV5) lac promoter and the stronger Trp(−35) lacUV5(−10) promoter were also constructed.

FIGS. 19A to 19C show the enhancement of hexose sugar production by rational metabolic engineering of Synechococcus. FIG. 19A shows the rationale for overexpression of UDP-glucose phosphorylase. UDP-glucose and fructose-6-P are the precursors of glucose and fructose in the artificial pathway for hexose production. UDP-glucose is produced by UDP-glucose phosphorylase, encoded by the galU gene. FIG. 19B shows the total glucose plus fructose concentrations in culture supernatants of induced Synechococcus glf+invA either with or without a galU expression construct. Dashed lines: glucose+fructose concentrations; solid lines: bacterial density. FIG. 19C shows the insertion of galU into “neutral site” NS3 with a chloramphenicol-resistance marker.

FIGS. 20A and 20B show the sequence for the CscB protein of E. coli. FIG. 20A shows the nucleic acid sequence and FIG. 20B shows the encoded amino acids.

FIGS. 21A and 21B show the sequence for the GltS protein of E. coli. FIG. 21A shows the nucleic acid sequence and FIG. 21B shows the corresponding amino acids.

### DETAILED DESCRIPTION

The present invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” References to SEQ ID NOs and the corresponding sequence listings are part of the present specification and fully incorporated herein.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The invention provides compositions and methods for the production of useful chemicals from photosynthetic microbes. It is an object of the invention to engineer photosynthetic microbes to synthesize and secrete useful organic chemicals. Many such organic compounds are hydrophilic and require transport proteins. Many photosynthetic microbes are incapable of transporting complex organic compounds because they lack many of the transporters found in other organisms such as E. coli.

Previous efforts to metabolically engineer cyanobacteria have focused on ethanol, isobutanol, and isobutyraldehyde, which are relatively lipophilic molecules that can directly cross cell membranes. Atsumi et al., 27 Nat. Biotechnol. 1177 (2009); Deng et al., 65 Appl. Environ. Microbiol. 523 (1999). One aspect of the invention described herein relates to engineering of photosynthetic bacteria to produce a much wider variety of compounds, using transporters to export the molecule of choice.

Many photosynthetic microbes have evolved to use only CO₂, fixed or atmospheric nitrogen, and various minerals. Photosynthetic microbes are ˜1 order of magnitude more productive than conventional terrestrial plants to capture solar energy and their photosynthetic efficiencies can be >10%. Huntley et al., 12 Mitigat. Adapt. Strat. Global Change 573 (2007); Li et al., 24 Biotech. Prog. 815 (2008). This self-sufficient mode of metabolism contrasts with that of E. coli, for example, which has a wide variety of transporters. Another point of contrast is that E. coli and many other microbes secrete organic compounds as waste products. Because of the high energetic cost of fixing carbon, photosynthetic microbes generally do not secrete carbon-based waste products. The present invention provides for photosynthetic microbes that are engineered to do so, including cyanobacteria, green sulfur bacteria (including Family Chlorobiaceae), purple sulfur bacteria (e.g., Family Chromatiaceae and Family Ectothiorhodospiraceae) purple nonsulfur bacteria (e.g., Family Rhodospirillaceae), and green bacteria (including Family Chloroflexaceae).

The genera of cyanobacteria that may be used in various embodiments include Chamaesiphon, Chroococcus, Cyanobacterium, Cyanobium, Dactylococcopsis, Gloeobacter, Gloeocapsa, Gloeothece, Microcystis, Prochlorococcus, Prochloron, Synechococcus, Synechocystis, Chroococcidiopsis, Cyanocystis, Dermocarpella, Myxosarcina, Pleurocapsis, Stanieria, Xenococcus, Arthrospira, Borzia, Crinalium, Geitlerinema, Halospirulina, Leptolyngbya, Limnothrix, Lyngbya, Microcoleus, Oscillatoria, Planktothrix, Prochlorothrix, Pseudanabaena, Spirulina, Starria, Symploca, Trichodesmium, Tychonema, Anabaena, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, Iyengariella, Stigonema, Rivularia, Scytonema, and Tolypothri.

Cyanobacteria are photoautotrophs, able to use CO₂ as their sole carbon source and light as their energy source. Unlike certain other photosynthetic bacteria, cyanobacteria use the same photosynthetic pathway as eukaryotic cells such as algae and higher plants (the “C3” or “Calvin” cycle). Other photosynthetic bacteria use different light-harvesting pigments (bacteriochlorophyll) and metabolic pathways. Particular exemplary cyanobacteria include Prochlorococcus, Synechococcus, and Synchecocystis. A number of cyanobacterial species have been manipulated using molecular biological techniques, including the unicellular cyanobacteria Synechocystis sp. PCC6803 and Synechococcus elongatus PCC7942, whose genomes have been sequenced completely.

In one embodiment aspect, the invention provides a genetically engineered photosynthetic bacteria (“host cell”) that expresses one or more heterologous enzyme and one or more heterologous transporter, wherein the transporter mediates the secretion of a product into the extracellular medium. In some embodiments, the product secreted by the recombinant bacterium is a chiral compound. Examples of chiral compounds include, without limitation, sugars and carboxylic acids, such as D-lactic acid and L-lactic acid. Exemplary sugars include lactose, galactose, glucose, fructose, a mixture of glucose and fructose, or sucrose. In another embodiment, chiral compounds include amino acids and vitamins.

Thus, in some embodiments, the transporter mediates the secretion of a product whose synthesis is enhanced by the heterologous enzyme. The heterologous enzymes can be of a type that catalyze a heterologous reaction that does not normally occur in the host organism, or can be an enzyme that is similar to an endogenous enzyme, but which catalyzes a desired reaction in a faster, more efficient, more specific, or otherwise preferable manner. For purposes of the invention, a “heterologous enzyme” is an enzyme that is not expressed naturally by the host cell. In some embodiments, heterologous enzymes include lactate dehydrogenase and invertase. For purposes of the invention, a “heterologous transporter” is a transporter that is not expressed naturally by the host cell.

In some embodiments, a gene encoding an enzyme that participates in a pathway that leads to the synthesis of a compound (such as an enzyme disclosed herein), is cloned into an expression vector for transformation into a photosynthetic bacterium. The vector includes sequences that promote expression of the transgene of interest, such as a promoter, and may include an intron sequence, a sequence having a polyadenylation signal, etc. Alternatively, if the vector does not contain a promoter in operable linkage with the gene of interest, the gene can be transformed into the cells such that it becomes operably linked to an endogenous promoter by homologous recombination or vector integration.

The heterologous genes may be “codon-optimized” for expression in an organism: the gene's nucleotide sequence has been altered with respect to the original nucleotide sequence such that one or more codons of the nucleotide sequence has been changed to a different codon that encodes the same amino acid, in which the new codon is used more frequently in genes of the host cell than the original codon. The degeneracy of the genetic code provides that all amino acids except for methionine and tryptophan are encoded by more than one codon. For example, arginine, leucine, and serine are encoded by different six different codons; glycine, alanine, valine, threonine, and proline are encoded by four different codons. Many organisms use certain codons to encode a particular amino acid more frequently than others. Without limiting any aspects of the invention to any particular mechanism, it is believed that some tRNAs for a given amino acid are more prevalent than others within a particular organism, and genes requiring a rare tRNA for translation of the encoded protein may be expressed at a low level due in part to a limiting amount of the rare tRNA. Thus, for adequate or optimal levels of expression of an encoded protein, a gene may be “codon-optimized” to change one or more codons to new codons (“preferred codons”) that are among those used more frequently in the genes of the host organism (referred to as the “codon preference” of the organism). As used in the context of the invention, a “codon-optimized” gene or nucleic acid molecule of the invention need not have every codon altered to conform to the codon preference of the intended host organism, nor is it required that altered codons of a “codon-optimized” gene or nucleic acid molecule be changed to the most prevalent codon used by the organism of interest. For example, a codon-optimized gene may have one or more codons changed to codons that are used more frequently that the original codon(s), whether or not they are used most frequently in the organism to encode a particular amino acid.

A variety of gene promoters that function in cyanobacteria can be utilized in expression vectors, including (a) the lac, tac, and trc promoters that are inducible by the addition of isopropyl β-D-1-thiogalactopyranoside (IPTG); (b) promoters that are naturally associated with transposon- or bacterial chromosome-borne antibiotic resistance genes (neomycin phosphotransferase, chloramphenicol acetyltrasferase, spectinomycin adenyltransferase, etc.); (c) promoters of various heterologous bacterial and native cyanobacterial genes; (d) promoters from viruses and phages; (e) salt-inducible promoters or other types of inducible promoters; and (f) synthetic promoters.

In some instances it can be advantageous to express a heterologous enzyme at a certain point during the growth of the transgenic host to minimize any deleterious effects on the growth of the transgenic organism and/or to maximize yield of the desired product. In these instances one or more exogenous genes introduced into the transgenic organism can be operably linked to an inducible promoter. The promoter can be a lac promoter, a tet promoter (e.g., U.S. Pat. No. 5,851,796), a hybrid promoter that includes either or both of portions of a tet or lac promoter, a hormone-responsive promoter (e.g., an ecdysone-responsive promoter, e.g., U.S. Pat. No. 6,379,945) a metallothionine promoter (U.S. Pat. No. 6,410,828), or a promoter that can be responsive to a chemical such as, for example, salicylic acid, ethylene, thiamine, or BTH (U.S. Pat. No. 5,689,044). An inducible promoter can also be responsive to light or dark (U.S. Pat. No. 5,750,385; No. 5,639,952) or temperature (U.S. Pat. No. 5,447,858; Abe et al., 49 Plant Cell Physiol. 625 (2008); Shroda et al., 21 Plant J. 121 (2000)), or copper level. Surzycki et al., 104 PNAS 17548 (2007). The promoter sequences can be from any organism, provided that they are functional in the host organism. Inducible promoters as used in the constructs of the present invention can use one or more portions or one or more domains of the aforementioned promoters or other inducible promoters fused to at least a portion of a different promoter that operates in the host organism to confer inducibility on a promoter that operates in the host species. Promoters isolated from cyanobacteria that have been used successfully include secA (controlled by the redox state of the cell); rbc (Rubisco operon); psaAB (light regulated); psbA (light-inducible); and nirA (NH₃/NO₃ regulated).

Likewise, a wide variety of transcriptional terminators can be used for expression vector construction. Examples of possible terminators include, but are not limited to, psbA, psaAB, rbc, secA and T7 coat protein, as are known in the art.

In some embodiments, a gene encoding an enzyme that participates in a pathway that leads to the synthesis of the chiral compound (such as an enzyme disclosed herein), is cloned into an expression vector for transformation into a photosynthetic bacterium. The vector includes sequences that promote expression of the transgene of interest, such as a promoter, an intron sequence, a sequence having a polyadenylation signal, etc. Alternatively, if the vector does not contain a promoter in operable linkage with the gene of interest, the gene can be transformed into the cells such that it becomes operably linked to an endogenous promoter by homologous recombination or vector integration.

Transformation vectors can also include a selectable marker, such as but not limited to a drug resistance gene, an herbicide resistance gene, a metabolic enzyme or factor required for survival of the host (for example, an auxotrophic marker), etc. Transformed cells can be optionally selected based upon the ability to grow in the presence of the antibiotic or other selectable marker under conditions in which cells lacking the resistance cassette or auxotrophic marker would not grow. In some embodiments a non-selectable marker may be present on a vector, such as a gene encoding a fluorescent protein or enzyme that generates a detectable reaction product. In an alternative transformation strategy, selectable or non-selectable markers can be provided on a separate construct, where both the gene-of-interest construct and the selectable marker construct are used together in transformation protocols, and selected transformants are analyzed for co-transformation of the construct that includes the gene-of-interest. See, e.g., Kindle, 87 PNAS 1228 (1990); Jakobiak et al., 155 Protist 381 (2004).

In some embodiments, a vector is designed for integration of the heterologous nucleic acid sequence into the host genome. Example vectors can be (a) targeted for integration into a bacterial chromosome by including flanking sequences that enable homologous recombination into the chromosome; (b) targeted for integration into endogenous host plasmids by including flanking sequences that enable homologous recombination into the endogenous plasmids; and/or (c) designed such that the expression vectors replicate within the chosen host. Artificial chromosome vectors can also be used for the transformation of photosynthetic microorganisms when more than one gene that encodes an enzyme that participates in the synthesis and/or a transporter that enables secretion of a product as described herein is transformed into an organism.

In some embodiments, the transporter is a member of the major facilitator superfamily. This family of proteins includes the Glut family of glucose transport proteins in humans and other mammals, the HXT family of glucose transporters in yeast, the lacY gene product, the lldP lactate transporter of E. coli or another bacterium that performs anaerobic metabolism, a mammalian citric acid transport protein, an ion-coupled sucrose transporter such as cscB, and the glpT glycerol phosphate transporter. The GLF protein of Z. mobilis is related to the glucose transporters from yeast and humans. These proteins have twelve membrane-spanning α-helices that form two rigid domains of six helices each. The subset of these proteins that includes the Glut, HXT and GLF proteins generally mediate transport of glucose, but their ability to transport other sugars such as fructose varies depending on the specific protein.

In one embodiment, photosynthetic bacteria are engineered to synthesize a mixture of glucose and fructose. This is achieved by the expression of a heterologous invertase enzyme that catalyzes the breakdown of sucrose into glucose plus fructose. Synthesis of sucrose within the cell can be induced in certain cyanobacteria such as S. elongatus 7942 and Synechocystis sp. 6803 by addition of salt to the medium; the sucrose is thought to maintain osmotic balance. Blumwald et al., 80 PNAS 2599 (1983). In alternative embodiments, the mechanism for producing sucrose utilizes key enzymes such as sucrose phosphate synthase and sucrose phosphate phosphatase expressed under the control of an inducible promoter with a different mode of regulation. In further embodiments, a heterologous hexose transporter is also expressed in the cyanobacteria, and this protein mediates the transport of glucose and fructose out of the cell.

When such a protein is co-expressed in cyanobacteria with invertase and sucrose synthesis is induced, the hexose products from the invertase reaction are secreted from the cell. For example, when the GLF and intracellular invertase proteins of Z. mobilis are expressed in S. elongatus 7942, intracellular sucrose is cleaved to glucose and fructose and these products are secreted into the medium.

In further embodiments, genetically engineered photosynthetic bacteria described herein for production of hexose sugars may be further optimized as follows. For example, it is sometimes useful to introduce a reduction-of-function mutation, such as a deletion, in an endogenous hexokinase gene, an endogenous glucokinase gene, or both. Without wishing to be bound by theory, this has the effect of reducing metabolism of glucose and/or fructose that is the desired product. In alternative embodiments, an additional gene that enhances production of UDP-glucose, which is a precursor of sucrose, can be further expressed in engineered photosynthetic microbes. For example, the galU gene that encodes a UDP-glucose pyrophosphorylase of a bacterium such as E. coli or a cyanobacterium can be used. Additional embodiments include over-expression of the genes that produce sucrose, which are sucrose phosphate synthase and sucrose phosphate phosphatase.

Another type of optimization relates to the type of promoter that is used for expression of the heterologous enzyme and/or transporter. In some embodiments, the enzyme is expressed from an inducible promoter. In another embodiment, the promoters used are selected from the lac operon promoter, a nitrogen-sensitive promoter, and a salt-inducible promoter. For example, the promoters for cyanobacterial nitrate reductase or nitrite reductase genes are repressed by ammonia and induced by nitrate; these are used as nitrogen-sensitive promoters.

It is convenient to use an inexpensive inducing agent, such as NaCl, rather than a more costly compound such as IPTG. Thus, NaCl-inducible promoters can be used for expression of the heterologous enzyme and/or transporter. The NaCl-inducible promoters for the cyanobacterial sucrose phosphate synthase and sucrose phosphate phosphatase genes can be used for this purpose.

In another embodiment, lactic acid is produced using the methods and organisms of the invention. In one embodiment, lactate dehydrogenase and a lactate transporter are expressed in a cyanobacterium. The lactate dehydrogenase (ldhA) can be derived from any organism, such as from Gram-negative organism such as E. coli, from another cyanobacterium, or from a mammal such as a human. For example, lactate dehydrogenase of E. coli or any of a wide variety of other organisms is expressed in a cyanobacterium, and a lactate/H⁺ co-transporter is also expressed. The E. coli lactate/H⁺ transporter illustrates many of the properties of this protein family. The E. coli protein is encoded by the lldP gene and is able to transport L-lactate, D-lactate, and glycolate, making it useful for transporting a variety of molecules from engineered organisms.

This lactate transporter protein, like the hexose transporter described above, is also in the major facilitator superfamily, of which members have twelve membrane-spanning α-helices, are thought to form two rigid domains of six helices each, and generally lack cleaved signal sequences. In the context of E. coli growing anaerobically, lactate is normally secreted as a waste product. The co-transport of a proton adds to the electrochemical gradient and export of lactate is thus an energy-generating step. When cyanobacteria are cultured, the pH of the medium often rises to pH 10 or pH 11 (Becking et al., 68 J. Geol. 243 (1960)). Thus, co-transport of lactate and a proton is strongly driven by the pH gradient in such cultures, and the reverse transport is negligible.

Another aspect of the invention provides for recombinant photosynthetic bacteria, such as cyanobacteria, that express a heterologous transporter that is coupled to the pH gradient across a cell membrane. In some embodiments, genetically engineered photosynthetic bacteria, such as cyanobacteria, can express a heterologous transporter that mediates the symport or antiport of a product and an inorganic ion. Examples of an inorganic ion include, but not limited to, a proton, a sodium ion, a potassium ion, a chloride ion, and a hydroxide ion. In additional embodiments, genetically engineered photosynthetic bacteria, such as cyanobacteria, can express a heterologous transporter that co-transports a proton with a metabolite. In further embodiments, the invention provides genetically engineered photosynthetic bacteria, such as cyanobacteria, that express a heterologous transporter that co-transports a sodium ion with a metabolite.

According to the invention, the principle of coupling transport to the pH gradient is generalized. Other bacteria express a number of transporters that, like the lldP protein, co-transport a given molecule with a proton or with a cation such as sodium that is coupled to the proton gradient. Such other bacteria generally secrete protons into the extracellular space, resulting in acidification of the medium. The secreted protons act as a form of stored energy, which is used when nutrient molecules are co-transported with a proton or sodium ion into the cell. Most cyanobacteria tend to alkalinize their growth medium, which differentiates them from other bacteria. Accordingly, such alkalinization will cause transporters that have evolved for nutrient import or energy generation to promote export of nutrients when the transporter is artificially expressed in a cyanobacterium. In some embodiments, the transporter can be a lactic acid/proton symporter, a sucrose proton symporter, a lactose/proton symporter, a dicarboxylic acid/proton symporter, an amino acid/proton symporter, a citrate/proton symporter, an amino acid/sodium ion symporter, or an amino acid/hydroxide ion antiporter. Examples of additional proton-coupled transporters include the sucrose transporter CscB (Vadyvaloo et al., 358 J. Mol. Bio. 1051 (2006)), the lactose permease of E. coli encoded by the lacY gene (Guan et al., 35 Ann. Rev. Biophys. Biomol. Str. 67 (2006)), and the mammalian H⁺/peptide transporter PepT1H (Chen et al., 272 Biochem. Biophy. Res. Commn. 726 (2000)).

Because cyanobacteria start with light and CO₂ as feedstocks, the cost of production of any carbon-based molecule is a function of the number of photons needed to drive the synthesis of the molecule and the efficiency of the engineered pathway. For example, the cost of lactic acid is currently higher than the cost of sugar because sugar is used as a feedstock to produce lactic acid. Without wishing to be bound by theory, production of lactic acid by cyanobacteria can abolish this distinction, as the rates of production of sugars and lactic acid can become comparable.

Another aspect of the invention provides for the expression of a soluble NAD(P)H transhydrogenase in photosynthetic microbes engineered to express lactate dehydrogenase or other enzymes that perform redox reactions. In addition embodiments, it is often useful to eliminate, for example by mutation, any membrane-associated NAD(P) transhydrogenase. Without wishing to be bound by theory, one rationale is that most lactate dehydrogenase enzymes use NADH, rather than NADPH, as a substrate. During photosynthesis, NADPH is directly produced from electrons that emerge from Photosystem I via ferredoxin and ferredoxin-NADP reductase. Thus, in photosynthetically active cells, levels of NADPH generally exceed levels of NADH. Expression of a soluble NAD(P)H transhydrogenase in sufficient quantities thus moves the balance of reducing equivalents between NAD and NADP toward equilibrium.

The lactate and glucose transporters with twelve membrane-spanning regions represent a large class of such transporters, termed the major facilitatory superfamily. Marger et al., 18 Trends Biochem. Sci. 13 (1993). The lactate and glucose transporters, and many other proteins in this family, do not have signal sequences and the mechanism by which the proteins insert into the membrane is unknown. The results obtained here indicate that members of this superfamily can be co-expressed with appropriate enzymes, providing export of the desired product.

As noted herein, a wide variety to photosynthetic bacteria can be manipulated using the methods of the present invention. For example, a heterologous over-expression system based on Rhodospirillum rubrum has been reported whereby proteins can be expressed under control of the regulatable promoters of the puh and puf operons.

The photosynthetic apparatus in Rhodospirillum is less evolved than Rhodobacter, in which a full length cDNA encoding yellow tail (Seriola quinqueradiata) growth hormone was cloned into an expression vector (under the lac promoter of E. coli) that contained a Rhodobacter-specific replicon. The resulting plasmid was introduced by transformation into the marine purple non-sulfur photosynthetic bacterium Rhodobacter sp. strain NKPB0021. The plasmid was maintained as an autonomous replicon and showed good stability in the absence of antibiotics. Burgess et al., 15 Biotech. Letts. 111 (1993).

Further regarding Rhodobacter, a photosynthetic variant was engineered to produce hydrogen in a light-independent manner by adding pyruvate lyase and formate lyase complex to a photosynthetic strain of Rhodobacter sphaeroides using E. coli conjugation. U.S. Patent Appl. Pub. No. 2010/0003734. A versatile Rhodobacter heterologous protein expression vector comprising a promoter nucleic acid sequence operable in a Rhodobacter, a nucleic acid sequence encoding an extended purification tag, a cloning cassette comprising a multiple cloning site and a selection marker has been described (WO 07/038746) and may be adapted for use in the present methods.

Another cloning system has been used to produce branched-chain alcohols via heterologous alcohol dehydrogenase in Synechococcus elongatus. More specifically, a construct combining Saccharomyces cerevisiae pyruvate decarboxylase gene and S. cerevisiae alcohol dehydrogenase gene cloned into S. elongatus PCC 7942. Also, a Lactococcus lactis KDCa gene was combined with S. cerevisiae ADH2 gene and transformed into S. elongatus cells as described by Golden and Sherman (158 J. Bacteriol. 36 (1984)). Additionally, a Lactococcus lactis KDCa gene was combined with S. cerevisiae ADH2 gene and transformed into S. elongatus cells as described by Golden and Sherman (1984). See U.S. Patent Appl. Pub. No. 2010/0151545.

Another cyanobacterium, Synechocystis PCC 6803, was transformed with plasmids encoding codon-modified S. cerevisiaePDC1 and ADH2 genes, as described by Zang et al. (45 J. Microbio. 241 (2007)); and the acetolactate synthase gene from Synechocystis sp. PCC 6803 was redesigned and cloned into wild-type Synechocystis as described by Zang et al., 2007. See U.S. Patent Appl. Pub. No. 2010/0151545.

Others have described introducing O₂-tolerant hydrogenase from purple sulfur photosynthetic Thiocapsa roseopersicina into cyanobacteria Synechococcus and Synechocystis using a commercial E. coli shuttle vector; and introducing O₂-tolerant hydrogenase from purple non-sulfur photosynthetic Rubrivivax gelatinosus CBS into cyanobacteria Synechococcus and Synechocystis. J. Craig Venter Institute, Rockville, Md.

Further regarding the cyanobacterium Synechococcus, the eicosapentaenoic acid (EPA) synthesis gene cluster from an EPA-producing bacterium, Shewanella sp. SCRC-2738, was cloned into a broad-host range vector and then introduced into the marine Synechococcus sp. NKBG15041c, by conjugation. The transgene was expressed and EPA produced in a variety of light-levels and temperatures. Yu et al., 35 Lipids 1061 (2000). Similarly, recombinant Synechococcus sp. PCC 7942 was constructed for the photosynthetic conversion of CO₂ to ethylene by inserting the ethylene-forming enzyme of Pseudomonas syringae. Sakai et al., 84 J. Ferment. Bioeng. 434 (1997). Synechococcus sp. PCC 7942 was also transformed with a recombinant plasmid harboring poly-(hydroxybutyrate) (PHB)-synthesizing genes from the bacterium Alcaligenes eutrophus. The transformant accumulated PHB in nitrogen-starved conditions, the PHB content held stable during a series of batch cultures, and the yield was increased by CO₂-enrichment. Suzuki et al., 18 Biotech. Lett. 1047 (1996); Takahashi et al., 20 Biotech. Lett. 183 (1998).

Another effort showed that the E. coli lacZ gene could be expressed in Synechococcus R2 PCC7942 both as a plasmid-borne form and integrated into the chromosome. A promoterless form of the lacZ gene was used as a reporter gene to make transcriptional fusions with cyanobacterial promoters using a shuttle vector system and also via a process of integration by homologous recombination. Synechococcus R2 promoter-lacZ gene fusions were used to identify CO₂-regulated promoters by quantitatively assessing the β-galactosidase activity under high and low CO₂ conditions, which detected several promoters induced under low CO₂ conditions. Scanlan et al., 90 Gene 43 (1990).

Several vectors have been described for the expression of recombinant genes in the cyanobacterium, Anacystis, including recombinant plasmids capable of integration into the chromosome of A. nidulans R2 (Elanskaya et al., 11 Molec. Genet. Microbio. Virol. 20 (1985)); a hybrid plasmid capable of transforming E. coli and A. nidulans (Friedberg & Seijffers, 22 Gene 267 (1983)); and specifically E. coli K12 and A. nidulans R2 (Kuhlemeier et al., 184 Mol. Gen. Genet. 249 (1981).

Regarding the nitrogen fixing cyanobacterium Anabaena sp. PCC7120, a recombinant strain was designed to constitutively express the Ca²⁺-binding photoprotein apoaequorin in a study of Ca²⁺ transients in response to heat and cold shock signaling. Torrecilla et al., 123 Plant Physiol. 161 (2000). Hence, the methods of the present invention can be applied to a wide variety of photosynthetic bacteria.

In some embodiments, photosynthetic bacteria expressing a heterologous enzyme and a heterologous transporter as described herein can be further engineered to express one or more genes for enhancing production of hydrophilic products. In one embodiment, the additional genes encode enzymes for enhancing synthesis of intracellular precursors. For example, the galU gene from a bacterium such as E. coli can be further expressed in engineered photosynthetic microbes to increase intracellular sucrose, which is the precursor for production of glucose and fructose. Alternatively, the udhA gene from a bacterium such as E. coli can be further introduced into engineered photosynthetic microbes to increase intracellular NADH, which is the reducing substrate for lactate dehydrogenase required for conversion of pyruvate to lactate. Alternative embodiments include reduction-of-function mutation, such as deletion of genes that causes a decrease in intracellular precursors or synthesized hydrophilic products via different cellular processes such as metabolism, degradation, post-modification, or co-binding with other products.

Still another aspect of the invention provides for a method for producing a hydrophilic product, the method comprising culturing a microbe according to the first aspect of the invention in the presence of light, CO₂, appropriate minerals and water, and obtaining said the hydrophilic product from a supernatant of the cultured microbe. In some aspects the method further includes providing an inducing agent to induce the expression of at least one heterologous gene expressed in photosynthetic microbe. In one embodiment, the heterologous gene encodes an enzyme or a transporter. In further embodiments, the invention presented herein contemplates an attractive and economically feasible strategy for biofuel production using cyanobacteria in photobioreactors.

In another aspect, the invention provides a method for producing a hydrophilic product, the method comprising culturing a microbe according to the second aspect of the invention in the presence of light and CO₂, appropriate minerals and water, and obtaining said the hydrophilic product from a supernatant of the cultured microbe. In some aspects the method further includes providing an inducing agent to induce the expression of at least one heterologous gene expressed in photosynthetic microbe. In one embodiment, the heterologous gene encodes an enzyme, a transporter or both.

In various embodiments, products can be purified from culture supernatants by standard techniques known to a skilled artisan. See, e.g., Ikeda, in 79 ADVANCES BIOCHEMICAL ENGIN./BIOTECH.: MICROBIAL PROD. OF AMINO ACIDS, 1-35 (Faurie & Thommel, eds., Springer-Verlag, Berlin Heidelberg, Germany (2003)). Sugars are purified by concentration and crystallization or simply obtained as sugar solutions with minor inorganic contaminants from the medium after filtration to remove bacteria. Organic acids such as succinate or citrate are purified by acidification of the culture supernatant, followed by precipitation/crystallization of the organic acid, according to standard procedures well-known in the art.

In some embodiments of the present invention may be defined in any of the following numbered paragraphs:

A recombinant photosynthetic bacterium comprising at least one heterologous enzyme and at least one heterologous transporter, wherein said transporter mediates the secretion of a product whose synthesis is enhanced by said enzyme.

The recombinant photosynthetic bacterium of paragraph [0084], wherein the transporter is a member of the major facilitator superfamily.

The recombinant photosynthetic bacterium of paragraph [0084], wherein the heterologous transporter encoded by a gene selected from the group consisting of: the Glut family of glucose transport proteins in humans and other mammals; the HXT family of glucose transporters in yeast; lacY; lldP lactate transporter of E. coli or another bacterium that performs anaerobic metabolism; a mammalian citric acid transport protein; the glpT glycerol phosphate transporter; and GLF of Z. mobilis.

The recombinant photosynthetic bacterium of paragraph [0084], wherein the enzyme is an invertase.

The recombinant photosynthetic bacterium of paragraph [0086] or [0087], further comprising a reduction-of-function mutation in an endogenous hexokinase gene, an endogenous glucokinase gene, or both.

The recombinant photosynthetic bacterium of any of paragraphs [0084] to [0088], wherein the product is a chiral compound.

The recombinant photosynthetic bacterium of paragraph [0089], wherein the chiral compound is a hexose sugar.

The recombinant photosynthetic bacterium of paragraph [0090], wherein the hexose sugar is glucose, fructose, or a mixture of glucose and fructose.

The recombinant photosynthetic bacterium of paragraph [0084], wherein the enzyme is lactate dehydrogenase and the transporter is a lactate transporter.

The recombinant photosynthetic bacterium of paragraph [0092], wherein the lactate transporter is encoded by the lldP gene of E. coli.

The recombinant photosynthetic bacterium of paragraph [0092] or [0093], further comprising a reduction-of-function mutation in malate dehydrogenase, pyruvate dehydrogenase complex, phosphoenolpyruvate carboxylase, phosphoenolpyruvate synthase.

The recombinant photosynthetic bacterium of any of paragraphs [0092] to [0094], wherein the product is lactic acid.

The recombinant photosynthetic bacterium of any of paragraphs [0084] to [0095], wherein the enzyme is expressed from an inducible promoter.

The recombinant photosynthetic bacterium of paragraph [0096], wherein the photosynthetic bacterium is a cyanobacterium.

The recombinant photosynthetic bacterium of paragraph [0097], wherein the cyanobacterium is selected from Prochlorococcus spp., Synechococcus spp., and Synchecocystis spp.

The recombinant photosynthetic bacterium of any one of paragraphs [0084] to [0098], wherein the photosynthetic bacterium is additionally engineered to express a soluble NAD(P) transhydrogenase.

The recombinant photosynthetic bacterium of any of paragraphs [0084] to [0098], wherein the photosynthetic bacterium is additionally engineered to express a UDP-glucose phosphorylase.

A recombinant photosynthetic bacterium comprising at least one heterologous transporter, wherein said transporter mediates the symport or antiport of a product and an inorganic ion.

The recombinant photosynthetic bacterium of paragraph [00101], wherein said inorganic ion is selected from the group consisting of a proton, a sodium ion, a potassium ion, a chloride ion, and a hydroxide ion.

The recombinant photosynthetic bacterium of paragraph [00101], wherein said transporter is selected from the group consisting of a lactic acid/proton symporter, a sucrose proton symporter, a lactose/proton symporter, a dicarboxylic acid/proton symporter, an amino acid/proton symporter, a citrate/proton symporter, an amino acid/sodium ion symporter, and an amino acid/hydroxide ion antiporter.

The recombinant photosynthetic bacterium of paragraph [00101], wherein the heterologous transporter encoded by a gene selected from the group consisting of: the sucrose transporter CscB; the lactose permease of E. coli encoded by the lacY gene; the mammalian H+/peptide transporter PepT1H; the glutamate transporter GltS and the lactate transporter lldP.

The recombinant photosynthetic bacterium of paragraph [00101], wherein the product is a chiral product.

The recombinant photosynthetic bacterium of paragraph [00101], wherein the product is an amino acid.

The recombinant photosynthetic bacterium of paragraph [00106], wherein the amino acid is glutamate.

The recombinant photosynthetic bacterium of paragraph [00101], wherein the product is a sugar.

The recombinant photosynthetic bacterium of paragraph [00101], further comprising at least one more heterologous gene.

The photosynthetic bacterium of paragraph [00109], wherein the heterologous gene encodes an enzyme or a transporter.

The photosynthetic bacterium of paragraph [00110], wherein the enzyme is ldhA.

A method for producing a product, said method comprising culturing a recombinant photosynthetic bacterium of any of paragraphs [0084] to [00100] in the presence of light and carbon dioxide, and obtaining the product from the supernatant of the cultured bacterium.

A method for producing a product, said method comprising culturing a recombinant photosynthetic bacterium of any of paragraphs [00101] to [00111] in the presence of light and carbon dioxide, and obtaining the product from the supernatant of the cultured bacterium.

### EXAMPLES

The following Examples are intended to further illustrate certain embodiments of the invention and are not to be construed as limiting the scope of the invention.

The sequences of the integrated transgenes used in the Examples have been deposited in the GenBank database under accession numbers HM026754 (ldhA in NS2), HM026755 (lldP in NS1), HM026756 (glf in NS1), HM026757 (invA in NS2), and HM026758 (novel transgene in NS3).

### Example 1

### Engineering of a Photosynthetic Bacterium for Production of Sugars

To engineer a photosynthetic microbe to produce the hydrophilic compounds, e.g., glucose and fructose or lactic acid, enzymes for intracellular synthesis of these hydrophilic molecules of interest and also relevant transporters for export of the synthesized hydrophilic compounds can be expressed in the photosynthetic microbe. In one embodiment, an engineered photosynthetic microbe that produces and secretes intracellular sugars, particularly glucose and fructose, was constructed as follows. The specific microbe used in this Example was the cyanobacterium S. elongatus PCC7942 (Synechococcus), although a wide variety of cyanobacteria and other microbes could be used. S. elongatus 7942 is a fresh water cyanobacterium whose growth is somewhat inhibited by salt. In response to salt water, e.g., NaCl treatment, S. elongatus 7942 synthesizes sucrose, which remains intracellular and serves to osmotically balance the inside of the cell with the extracellular medium. Blumwald et al., 80 PNAS 2599 (1983); Miao et al., 218 FEMS Microbio. Lett. 71 (2003).

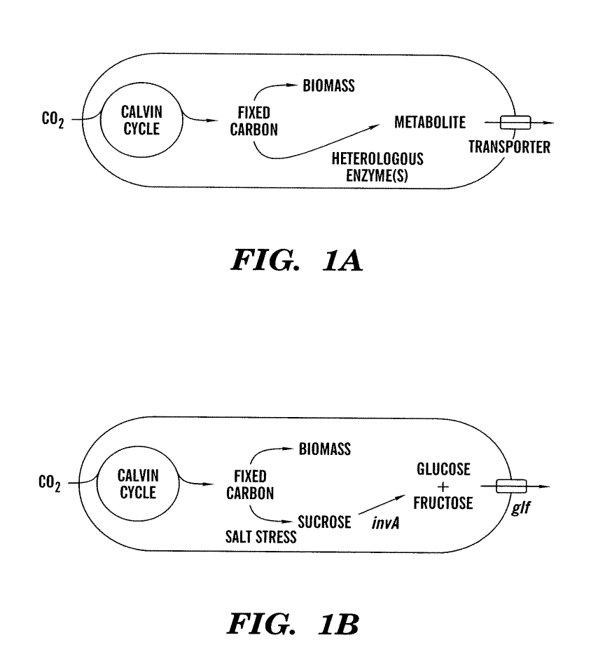

Engineering strategy: To engineer recombinant S. elongatus 7942 that produce and secrete a mixture of glucose and fructose, two genes were introduced into S. elongatus 7942: (a) the Zymomonas mobilis invA gene encoding a soluble, cytoplasmic invertase (Yanase et al., 55 Agric. Biol. Chem. 1383 (1991)) to cleave NaCl-induced sucrose, and (b) the Z. mobilis glf gene encoding a glucrose- and fructose-facilitated diffusion transporter, which allows export of the sugars from the cell. Barnell et al., 172 J. Bacteriol. 7227 (1990). The GLF protein belongs to the major facilitator superfamily whose members have twelve transmembrane α-helical segments and generally lack cleaved signal sequences. DiMarco et al., 49 Appl. Environ. 151 (1985); Snoep et al., 176 J. Bacteriol. 2133 (1994). The GLF protein facilitates diffusion of glucose and fructose in either direction across the membrane, and the transported sugar is not phosphorylated. Marger et al., 18 Trends Biochem. Sci. 13 (1993). According to an aspect of the invention, when S. elongatus 7942 is placed in a high-salt environment and the invA and glf genes are expressed, the bacterium produces intracellular sucrose, which is then cleaved into glucose and fructose by the invertase protein and allowed to be transported across the cell membrane into the medium by the hexose transporter encoded by glf. This general concept is illustrated in FIG. 1.

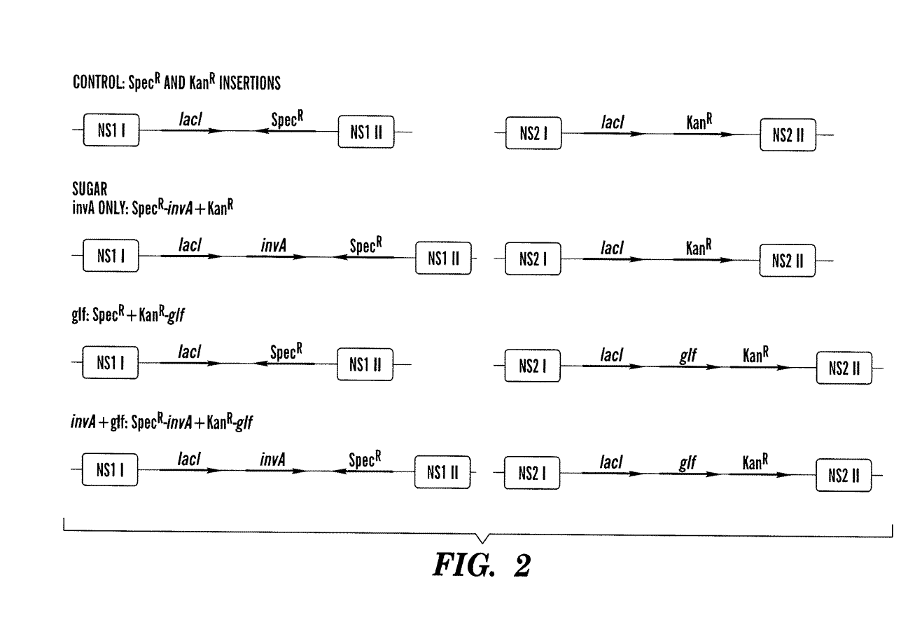

Plasmid and strain construction: The heterologous invA and glf genes were inserted into plasmid vectors that mediate integration into the S. elongatus 7942 genome by homologous recombination using neutral sites (Clerico et al., 362 Methods Mol. Biol. 155 (2007)) that can tolerate insertion with no phenotypic effects (Clerico et al., 362 Mets. Mol. Bio. 155 (2007); Mackey et al., 362 Mets. Mol. Bio. 15 (2007)). Neutral site 1 (NS1) and NS2 are present in plasmids DS1321 and DS21, which confer spectinomycin and kanamycin resistance, respectively, and contain E. coli lad and an isopropyl-β-D-thiogalactopyranoside (IPTG)-regulated trp-lac strong promoter. Diagrams of these vectors are shown in FIG. 2.

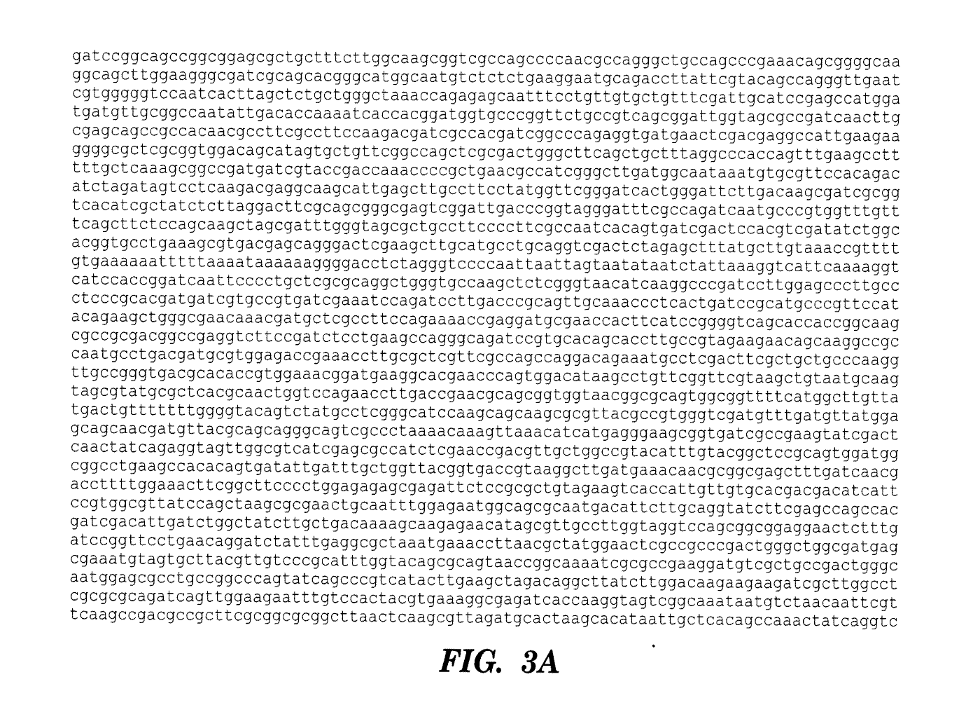

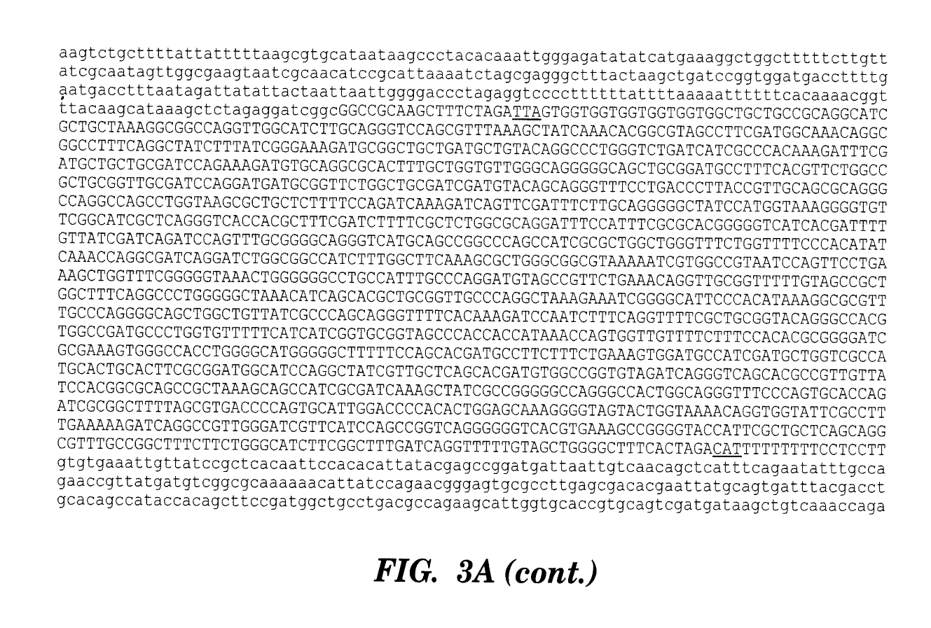

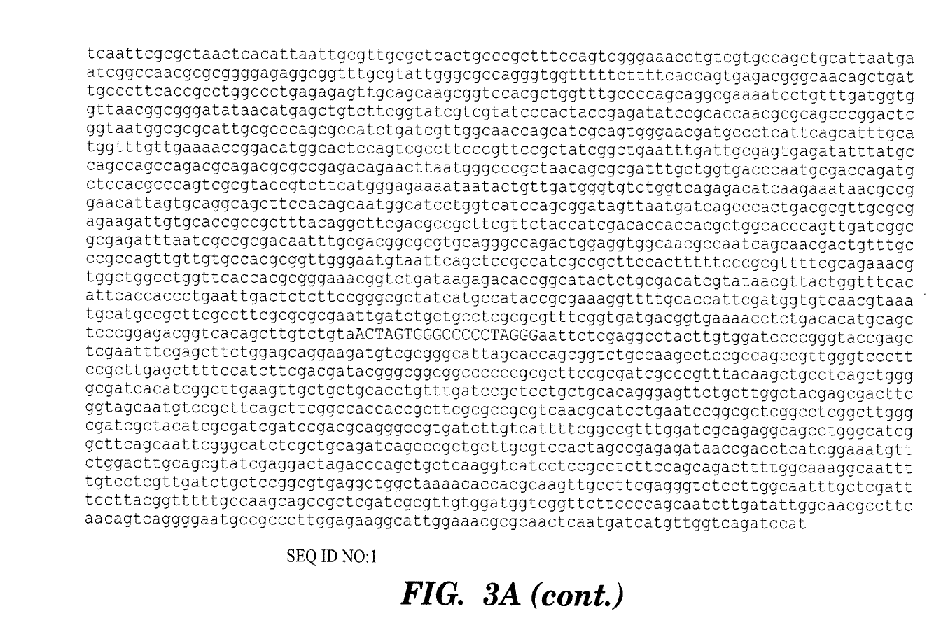

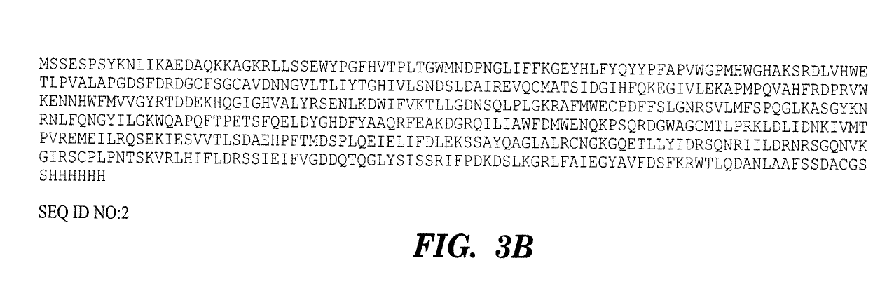

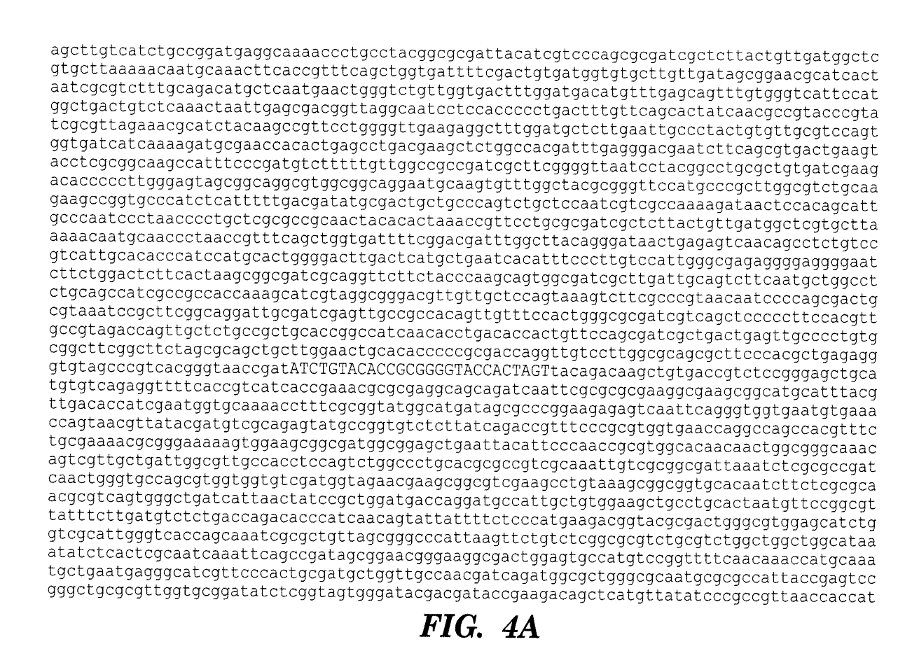

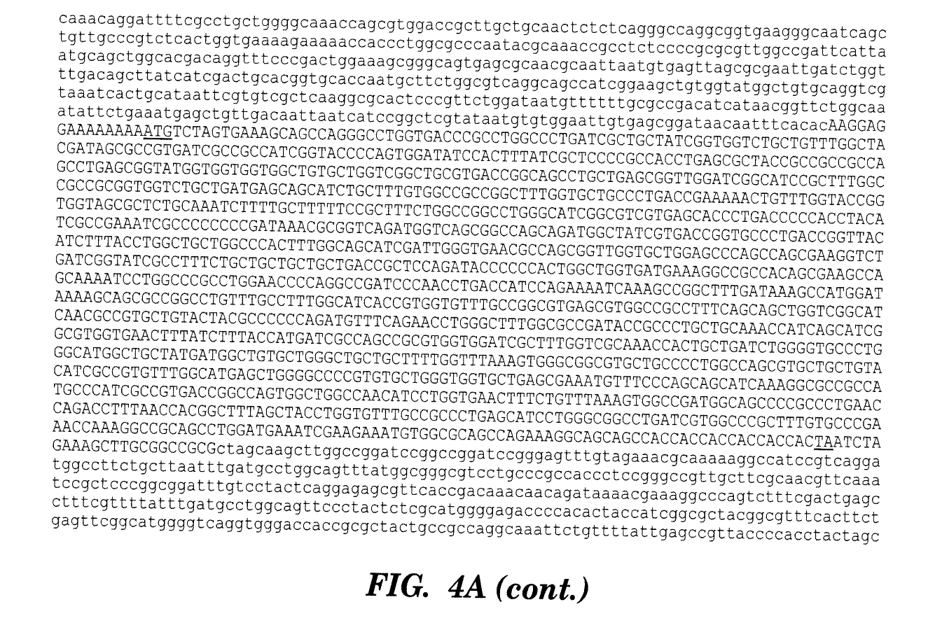

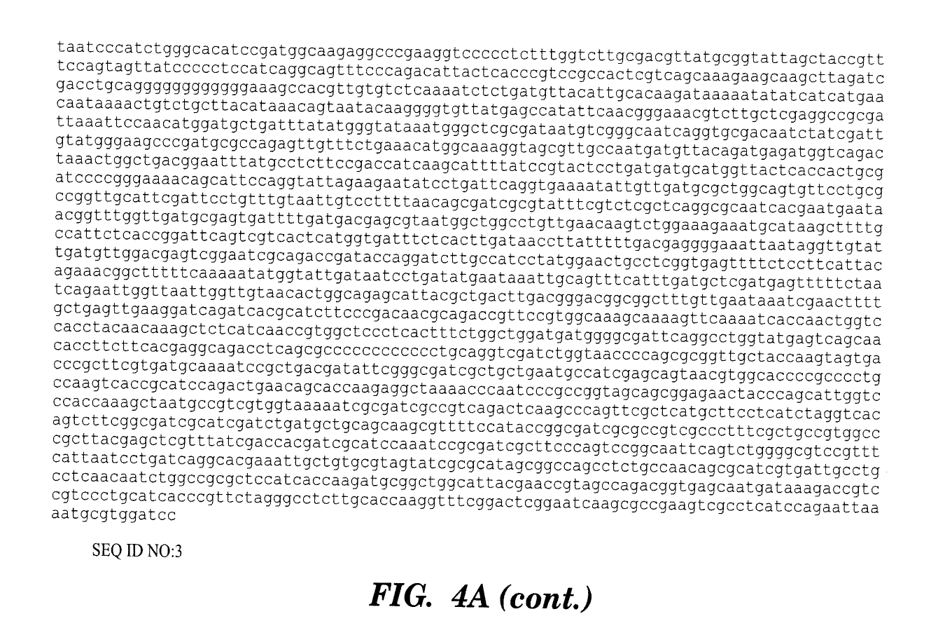

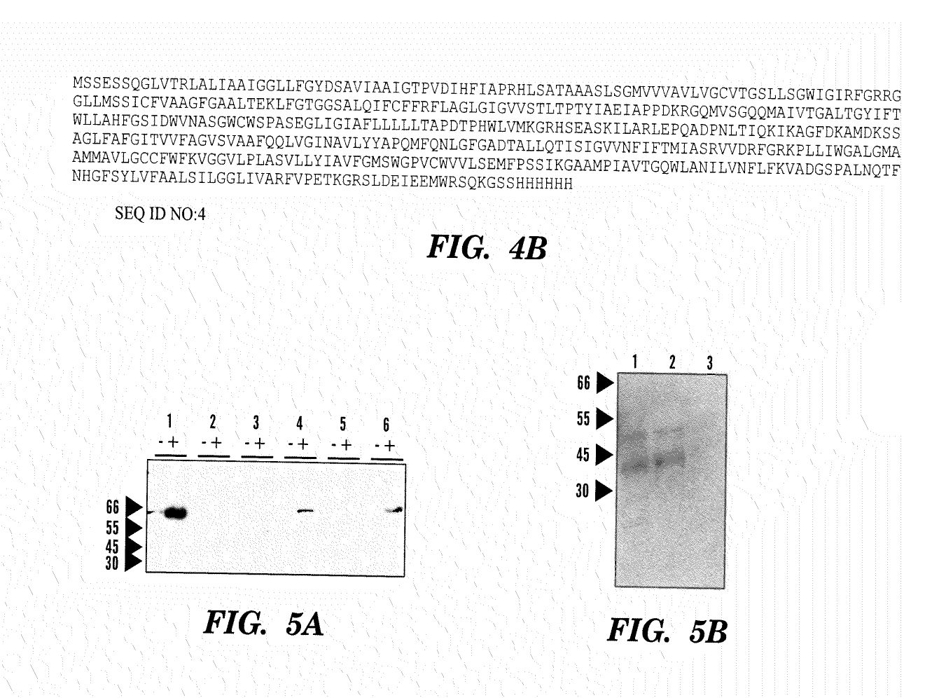

The invA and glf genes from Z. mobilis were obtained from Genscript (Piscataway, N.J.). The two genes were codon-optimized for expression in Synechococcus and were synthesized so that they contained a C-terminal His₆ tag. These genes were inserted into DS21 and DS1321 by using standard procedures. The invA gene was inserted in NS 1 along with a spectinomycin-resistance marker and a lad gene; the invA gene was expressed from an E. coli trp/lac promoter and also encoded a C-terminal His₆ tag as an epitope for detection in western blots. The glf gene was inserted in NS 2 along with a kanamycin-resistance marker and a lad gene; the glf gene was expressed from an E. coli lac promoter and also encoded a C-terminal His₆ tag as an epitope for detection in western blots. The sequences of these regions are shown in FIGS. 3 and 4, respectively. The E. coli lad gene was also expressed in Synechococcus from its own promoter. Transformation of Synechococcus was performed as described previously. Clerico et al., 362 Mets. Mol. Bio. 155 (2007). Integration of vectors into neutral sites was verified by PCR to demonstrate the presence of appropriate novel chromosome-transgene junctions and the absence of uninserted sites.

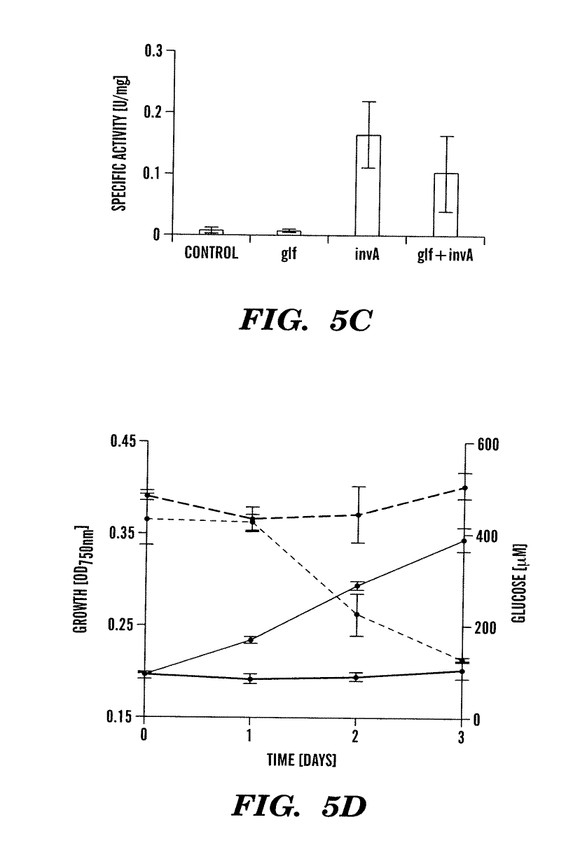

Activity of transgenes in vivo: Accordingly, strains of S. elongatus 7942 were constructed that express invA and glf, invA alone, glf alone, or neither transgene. To control for the presence of antibiotic resistance genes and lad genes and to allow use of the same antibiotics in cultures being compared, ‘empty’ insertions containing only these genes were made in some strains. The expression and functions of heterologous genes were determined by performing direct assays, as well as by examining secretion of molecules of interest in engineered Synechococcus strains. The functionality of the cloned Z. mobilis invA and glf genes in E. coli was shown as follows: the glf expression plasmid rescued growth of an E. coli ptsI mutant (CHE30) (Plumbridge, 33 Mol. Microbio. 260 (1999)) on glucose, and E. coli DH5α containing the invA expression plasmid was able to use sucrose as a carbon source. Expression of InvA-His₆ in Synechococcus was observed by western blotting and was IPTG-inducible, although GLF-His₆ were not detected (FIG. 5A), perhaps due to low levels of expression combined with poor extraction from membranes or His₆ tag being proteolytically removed. The invertase activity in Synechococcus extracts was increased ˜18-fold by expression of invA (FIG. 5C). The functionality of glf in Synechococcus was shown by adding 500 μM glucose or fructose to the culture medium, which allowed slow, heterotrophic growth of the cyanobacteria in the dark, while the sugar disappeared from the medium (FIG. 5D).

Experimental method for invertase activity assay: Crude cell extracts of Synechococcus strains expressing invA and glf were prepared from a culture that had been induced with 100 μM IPTG for 3 days. Cell pellets were resuspended in invertase assay buffer and disrupted by sonication on ice for 2.5 sec followed by a 5-sec break, using a total sonication time of 5 min. Cell debris was removed by centrifugation. Invertase activity was measured as described (Yanase et al., 55 Agric. Biol. Chem. 1383 (1991)), using an assay mixture containing crude cell extracts in 100 mM sodium acetate buffer (pH 5) at 30° C. The reaction was started by addition of 150 mM sucrose. After 10 min incubation, the reaction was stopped by incubation at 100° C. for 2 min. The heat-denatured proteins were pelleted, and the glucose concentration in the supernatant was determined using a sucrose-glucose-fructose assay kit (Megazyme Intl., Wicklow, Ireland). To determine the invertase specific activity in the crude cell extracts, the protein concentration was measured by performing a Bradford protein assay (Bio-Rad).

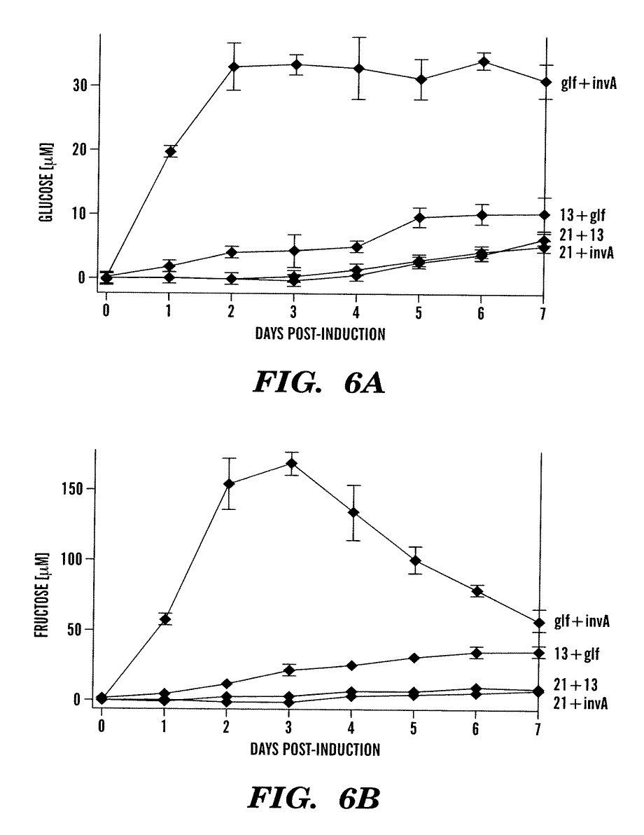

Secretion of sugars from engineered cynaobacteria: Coexpression of catabolic enzymes and transporters led to secretion of hexose sugars. Typical results are shown in FIGS. 6, 7 and 8. These results are from a representative experiment in which on Day 0, engineered Synechococcus containing the invA and glf transgenes was treated with 200 mM NaCl to induce sucrose synthesis and 100 micromolar IPTG to induce expression of the invA and glf genes. Cells were grown in alternating 18 hours of light, 6 hours of dark, in atmospheric levels of CO₂ at 30° C. The strains tested were a control strain carrying “empty vector” chromosomal insertions, strains carrying the glf gene or the invA gene with empty insertions corresponding to the other gene, and a strain carrying both the glf and invA genes. These strains are schematically shown in FIG. 2.

FIG. 6 shows that the Synechococcus strain expressing the invA and glf genes produced much larger amounts of glucose and fructose than the other strains, and the maximum total concentration was about 200 μM when both invA and glf genes were expressed (FIGS. 6A-6B). Thus, the combination of both the invA and glf genes is particularly useful for production of glucose and fructose by photosynthetic bacteria.

The glucose levels in the medium were significantly lower than the fructose levels, and the fructose levels declined after reaching a maximum level as the cell density increased (FIGS. 6A and 6B); these observations suggest that the engineered cyanobacteria could re-transport glucose and fructose back into the cell and metabolize them. This idea is further supported by observations below. According to the invention, the yield of glucose and fructose is further improved by the introduction of mutations such as reduction-of-function mutations in hexokinase and glucokinase.

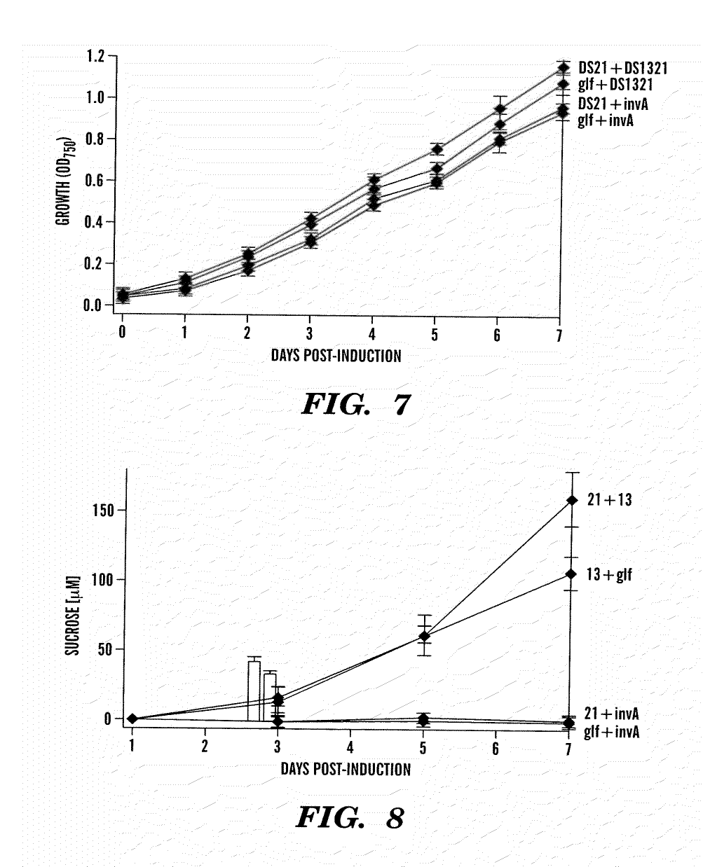

In this experiment, over 10% of the metabolic capability of the cells initially appeared to be directed toward secreted hexose production. Total hexose in the medium increased up to day 2 and then leveled off. On day 2, the total dry weight of the engineered cyanobacteria was about 280 μg/ml, and the total mass of hexose in the medium was about 33 μg/ml. Further, FIG. 7 shows the exponential growth rates of the four strains, and illustrates that the strain carrying neither transgene grew the fastest (0.80±0.002 per day), the strain earring the glf gene grew slightly more slowly (0.74±0.01 per day), and the strains carrying the invertase (0.61±0.01 per day) or the invertase gene and the glf gene grew the slowest (0.70±0.04 per day), but these growth rates were not dramatically different. This indicates that under the conditions employed, sugar production did not lead to a major diversion of the carbon flux.

FIG. 8 shows the amount of sucrose produced by the engineered cyanobacteria. The levels of sucrose in the medium were low during the initial phase of the experiment. FIG. 8 shows that the extracellular sucrose was not detected in strains expressing invA only, or a combination of invA and glf, and there were not significant differences between the wild-type and glf-expressing Synechococcus strains.

Further, the combined extracellular and intracellular sucrose concentration was measured in the engineered cyanobacteria by sonicating a sample of the whole culture on day 3, including supernatant, prior to removal of cell debris by centrifugation. FIG. 8 shows that no sucrose was observed from invertase-expressing strains. The amount of sucrose inside the wild-type and glf-expressing Synechococcus strains significantly exceeded the amount in the medium as compared with FIG. 8. In addition, the moles of fructose in the medium secreted by the engineered cyanobacteria significantly exceeded the total moles of sucrose found in the wild-type strain and in the respective medium, as compared with FIG. 6B.

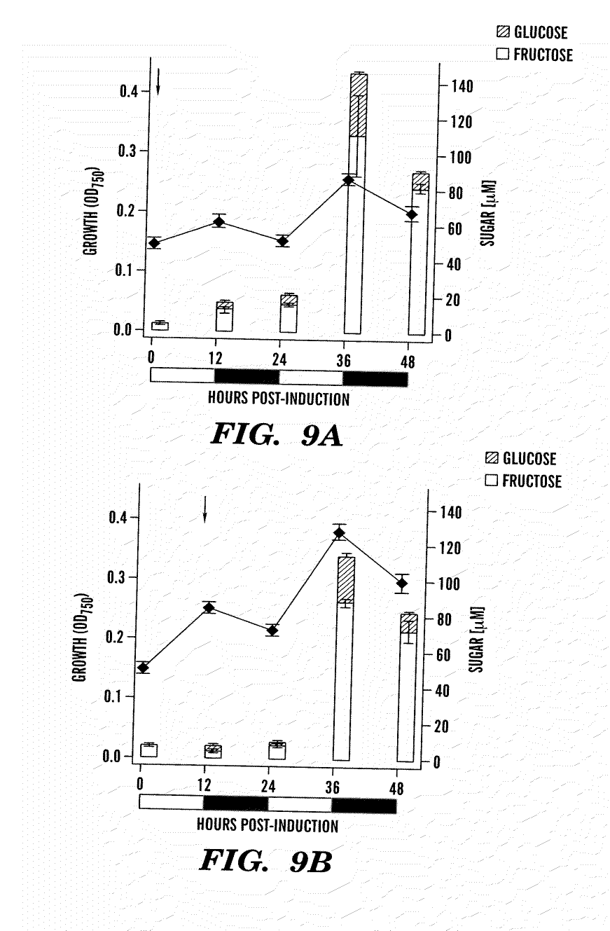

Cyanobacterial cultures maintained with alternating dark and light periods accumulate polysaccharides when they are exposed to light and mobilize this intracellular reserve material in the dark. Smith, 134B Ann. Microbiol. 93 (1983). To address whether induced sugar synthesis occurs during the day or whether stored energy reserves are used during the night, the growth and fructose and glucose sugar secretion of Synechococcus expressing glf and invA were monitored as a function of the day-night cycle consisting of alternating 12 hours of light and12 hours of darkness. Increases in the cell mass and net hexose sugar secretion occurred only in the presence of light; both parameters decreased in the dark (FIGS. 9A and 9B). This observation indicates that sucrose is synthesized during the day, and illustrates how engineered secretion systems can be used to noninvasively assay metabolic states.

In this Example, the following conditions and methods were used for culture of S. elongatus 7942. All Synechococcus strains were cultured at 30° C. in BG-11 medium (Allen et al., 51 J. Gen. Microbio. 199 (1968)), the formula for which is available from the American Type Culture Collection (ATCC, Manassas, Va.). Wild-type S. elongatus 7942 was obtained from the ATCC. Unless indicated otherwise, cultures were grown with a light cycle consisting of 18 hr of light and 6 hr of darkness. To select for homologous recombination and integration of heterologous DNA into the genome and for culture maintenance of engineered strains, kanamycin (25 μg/ml or 2.5 μg/ml if in combination with other drugs), spectinomycin (25 μg/ml or 2μg/ml if in combination with other drugs) was added to the medium. For all experiments, Synechococcus cultures were tested for possible contamination by spotting cultures onto LB medium plates; only data obtained with uncontaminated cultures are reported herein. Alternative concentrations of antibiotic concentrations, when used singly, were 50 μg/ml for kanamycin and 40 μg/ml for spectinomycin.

For sugar production experiments, liquid cultures were inoculated with exponentially growing cells (e.g. cells from a 2-3 day old dense culture) at an optical density of 750nm (OD₇₅₀) of 0.05. Sugar production was induced with 200 mM NaCl and 100 μM IPTG. Alternative concentrations of NaCl for induction of sugar production range between 100 to 300 mM (in Example 2). Growth was monitored by measuring the OD₇₅₀. Sugar production was determined using a sucrose-D-fructose-D-glucose assay kit (catalogue number KSUFRG; Megazyme, Ltd., Bray, Ireland). Assays were performed using culture supernatants prepared by centrifuging samples for 5 min at 21,130×g. Assays were performed in triplicate, and standard deviations were determined.

### Example 2

### Production of Hexoses as a Function of Salt Concentration

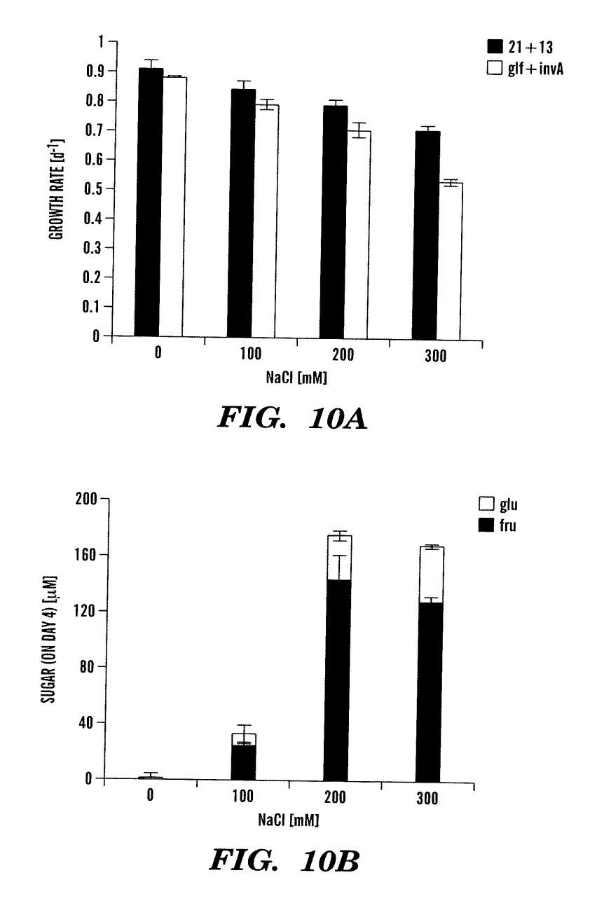

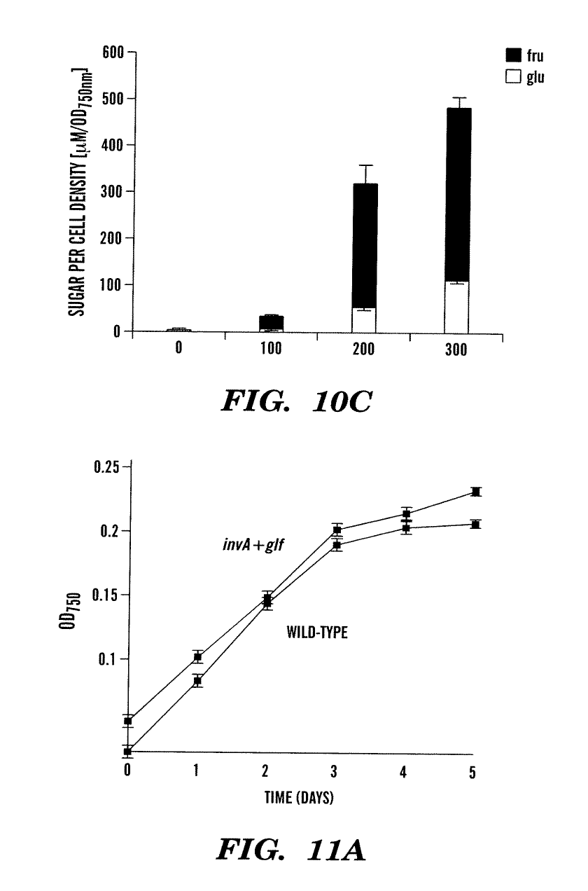

As the NaCl concentration determines how much sucrose accumulates inside the cells (Li et al., 24 Biotech. Prog. 815 (2008)), the effect of different salt concentrations on hexose sugar secretion by invA+glf-expressing Synechococcus was determined. Table 1 shows growth rates of a Synechococcus empty vector control and glf+invA strain, and sugar concentrations in the culture medium four days after induction with 100 mM IPTG and various different NaCl concentrations in BG-11 medium. FIGS. 10A-10C are graphical representations of data shown in Table 1.

TABLE 1
Effect of NaCl concentration on the growth rate
and hexose sugar secretion

| | | Sugar concentration [μM] | |
|---|---|---|---|
| NaCl [mM] | growth rate [d⁻¹] | Glucose | fructose |
| control | | | |
| 0 | 0.91 ± 0.03 | 0.1 ± 0.1 | 0.7 ± 0.2 |
| 100 | 0.84 ± 0.03 | 1.1 ± 0.5 | 0.9 ± 0.3 |
| 200 | 0.79 ± 0.02 | 0.7 ± 0.4 | 2.3 ± 1.9 |
| 300 | 0.71 ± 0.01 | 1.4 ± 0.2 | 0.7 ± 0.2 |
| glf + invA | | | |
| 0 | 0.88 ± 0.001 | 2.2 ± 2.5 | 0 ± 0.6 |
| 100 | 0.79 ± 0.02 | 8.6 ± 6.3 | 25.1 ± 2.0 |
| 200 | 0.71 ± 0.02 | 31.3 ± 3.6 | 144.4 ± 18.2 |
| 300 | 0.53 ± 0.01 | 40.0 ± 1.5 | 128.9 ± 4.5 |

With increasing salt concentrations, growth rates of the engineered strain decreased, and the inhibitory effect on growth of the glf+invA-expressing strain was more pronounced than the inhibition observed with a control strain (Table 1 and FIG. 10A). In other experiments, use of 600 mM NaCl resulted in complete inhibition of growth of the glf+invA-expressing strain. The amounts of sugar produced were also dependent on the salt concentration, and highest for 200-300 mM NaCl (Table 1 and FIG. 10B). Further, FIG. 10C illustrates that increasing NaCl concentrations up to 300 mM resulted in an increasing level of hexose secretion, as calculated on a per cell basis.

On day 2 after induction of sugar secretion by 300 mM NaCl, sugar yield was about 30% of the biomass produced by the corresponding empty vector control under identical growth conditions.

### Example 3

### Utilization of Cyanobacterially Secreted Hexoses by a Second Bacterium

In some metabolic engineering applications, it is useful to perform some metabolic transformations in one organism and other metabolic transformations in a second. For example, it is relatively less convenient to engineer cyanobacteria for certain metabolic transformations than to use, for example, E. coli. The cost of the carbon source in commercial fermentation (e.g., E. coli) is significant: as much as 30% to 50% of the overall operating cost (Galbe et al., 108 Adv. Biochem. Eng. Biotech. 303 (2007)). Therefore, the ability of hexose-secreting cyanobacteria to support growth of E. coli can be valuable.

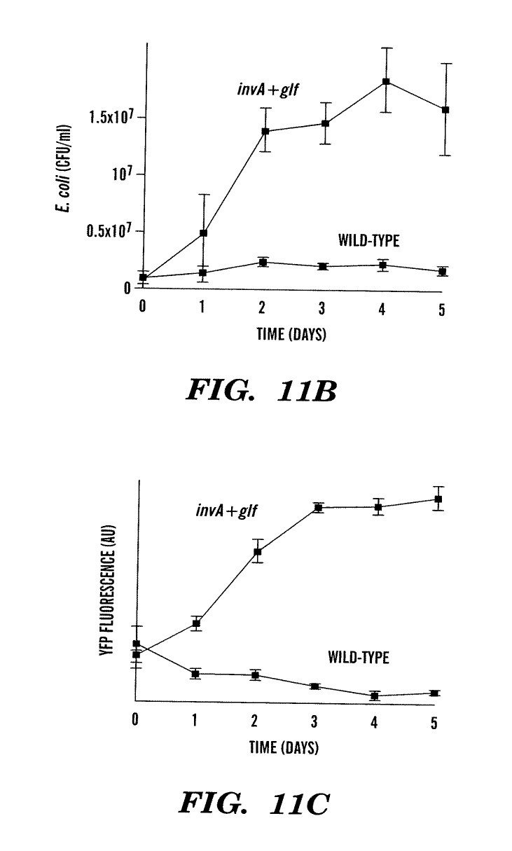

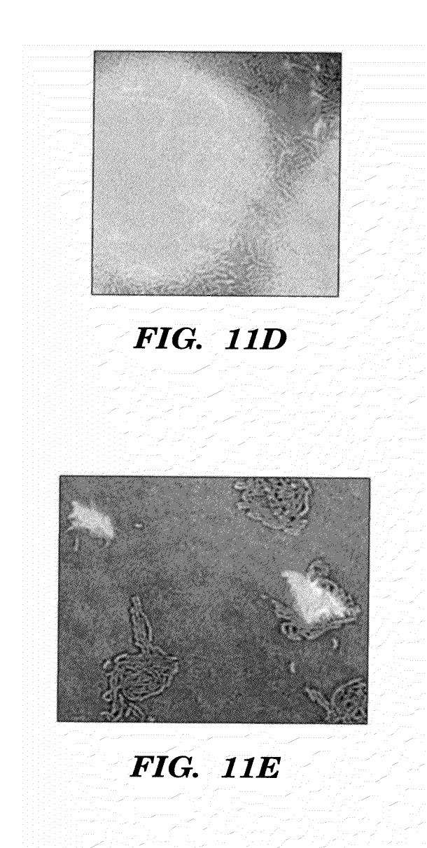

Typical results are shown in FIGS. 11A-11E. About 10⁶ cells/ml of a prototrophic strain of E. coli that expressed the yellow fluorescent protein (YFP) were added to cultures of a g/f+invA-expressing strain of S. elongatus 7942 or a control strain at an OD₇₅₀ of 0.1. At the time of addition of the E. coli, 100 micromolar IPTG and 200 mM NaCl were also added to induce hexose secretion. FIG. 11A illustrates that both cyanobacterial strains continued to grow in the presence of the E. coli. FIGS. 11B-11C illustrates that the E. coli were able to increase in number in the presence of the glf+invA-expressing strain of S. elongates 7942, but not in the presence of the control strain of S. elongatus 7942. These results indicate that nutrient-secreting cyanobacteria can be used to support the metabolism of a second microbe (e.g., E. coli) without addition of an external carbon source in liquid culture (FIGS. 11B and 11C) or on solid medium (FIGS. 11D and 11E). Without wishing to be bound by theory, coculture of sugar-secreting cyanobacteria and a second engineered microbe can allow production of a desired product without a reduced-carbon feedstock in situations where synthesis of the product is incompatible with cyanobacterial metabolism.

Experimental methods: Cultures of the Synechococcus glf+invA strain and a non-sugar-secreting control strain were grown to an OD₇₅₀ of 0.1. Sugar secretion was induced with 200 mM NaCl and 100 μM IPTG. E. coli cells from an overnight culture (LB+Kan, Spec+100 μl IPTG) were washed in PBS three times and incubated with shaking in PBS containing 100 μM IPTG and antibiotics for 1 hr at 37° C., after which 10⁶ cells/ml (30 μl) were added to 30 ml of either glf+invA or the empty vector control Synechococcus cultures. The medium used was BG-11 medium with 100 μM IPTG, 2.5 μg/ml kanamycin, 2 μg/ml spectinomycin, and 1 mg/ml NH₄Cl. The E. coli strain carried a plasmid that was a hybrid of the pET47b (Kan), with a Spectinomycin-Resistance gene and a promoter driving expression of YFP. Thus, the E. coli was also resistant to antibiotics used in the culture. Growth of E. coli was monitored by plating serial dilutions on LB agar and measuring the YFP fluorescence of culture samples with a Victor 3V plate reader. For microscopic quantitation of Synechococcus and E. coli, bacteria in samples of the liquid coculture were visualized by red chlorophyll autofluorescence and YFP fluorescence, respectively. To observe microcolony formation, 100 μl of the initial coculture was plated on BG-11 agar containing the compounds listed above. After four days, pieces of agar were cut out and placed onto a MatTek glass bottom dish for microscopy.

### Example 4

### Production of Lactic Acid from a Photosynthetic Microbe

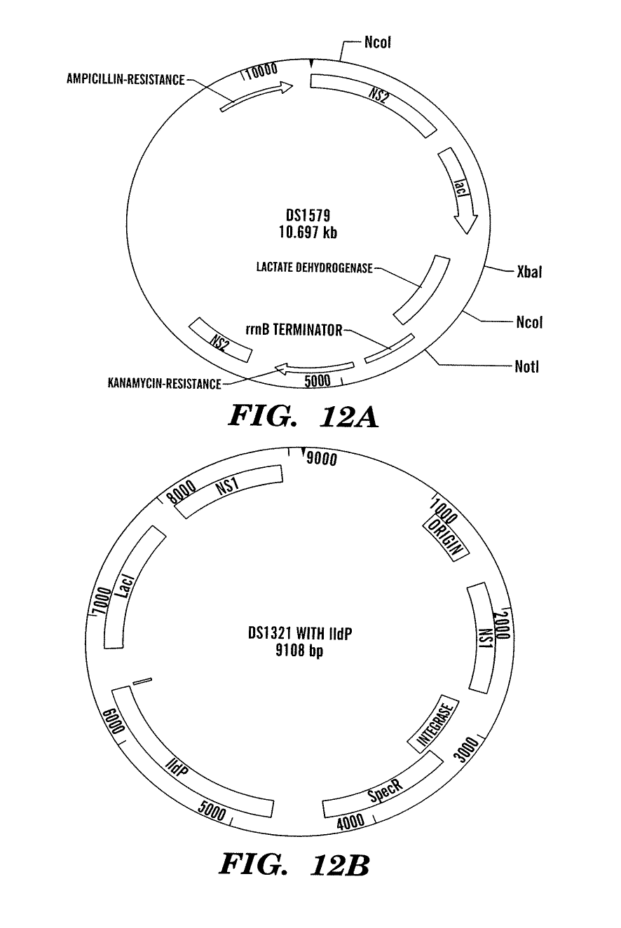

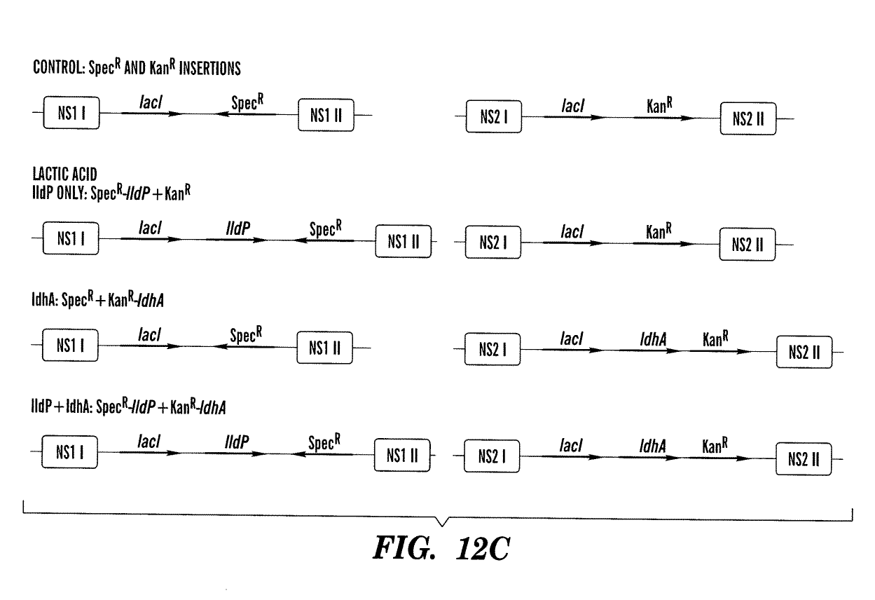

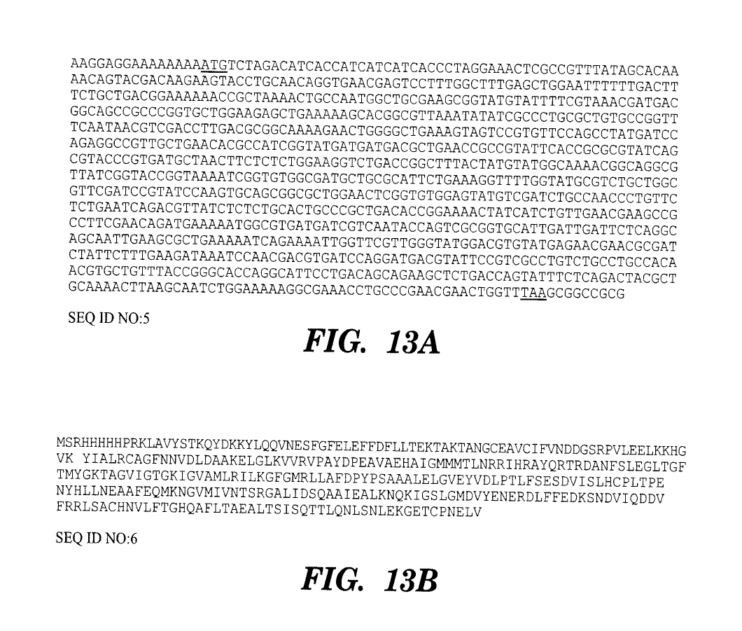

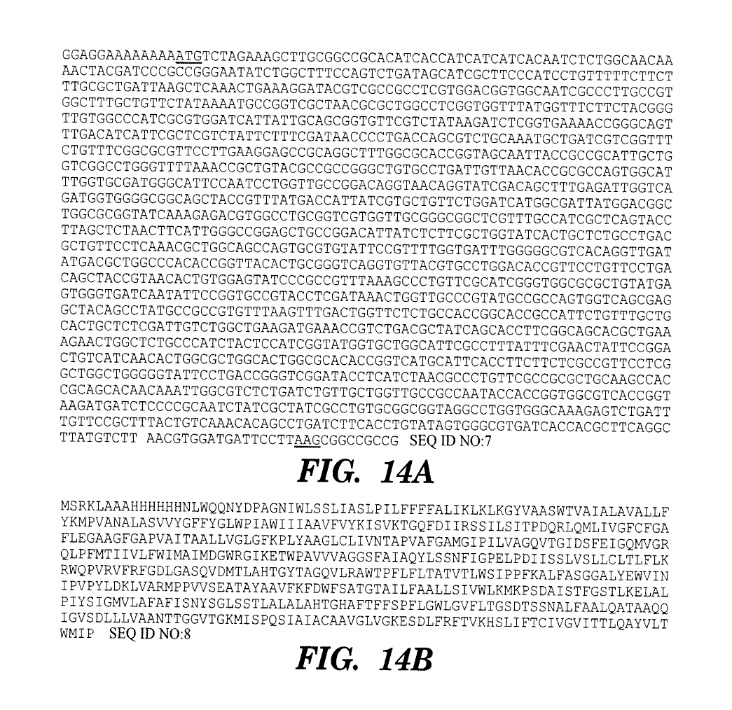

Using a similar approach to that was described in Example 1 for engineering of a photosynthetic bacterium to produce hexose sugars, a cyanobacterial strain was engineered to produce lactic acid. Specifically, lactate dehydrogenase ldhA from E. coli K12 (Plumbridge, 33 Mol. Microbio. 260 (1999)) was inserted into the DS21 plasmid, and the D,L-lactate transporter gene lldP from E. coli K12 (Nunez et al., 290 Biochem. Biophys. Res. Commun. 824 (2002)) was inserted into the DS1321 plasmid. Diagrams of these plasmid vectors for expression of ldhA and lldP in S. elongatus are shown in FIGS. 12A-C, and the sequences of the inserted ldhA and lldP genes with their respective amino acid sequences are shown in FIGS. 13A-B and 14A-B, respectively.

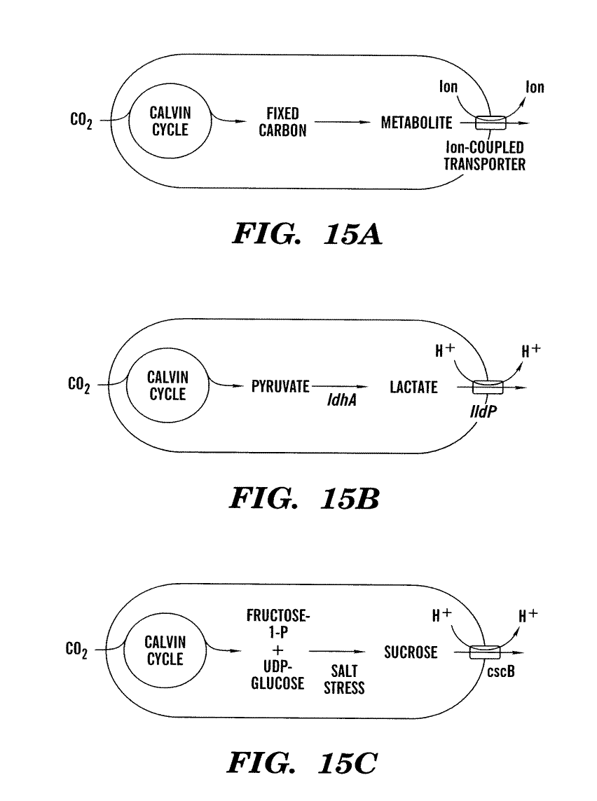

Synechococcus naturally produces pyruvate as a metabolic intermediate. The E. coli ldhA gene encodes lactate dehydrogenase, which catalyzes the reduction of pyruvate to lactate (Plumbridge, 1999), and the E. coli lldP gene encodes a lactate transporter protein in the major facilitator superfamily. DiMarco et al., 1985; Snoep et al., 1994. The LldP protein cotransports lactate with a proton (Nunez et al., 2002); during Synechococcus growth in BG-11 medium, when the culture reaches an OD₇₅₀ of 1, the pH of the medium is generally about pH 9, so lactate will be exported from the cell if it is produced intracellularly. The schematic depiction of this engineering scheme is illustrated in FIG. 15.

The ldhA and lldP were obtained by PCR amplification from E. coli DH5α using oligonucleotides that resulted in molecules with an N-terminal His₆ tag. To verify the activity of transgenes in the engineered cyanobacteria, expression of LdhA-His₆ in Synechococcus was observed by western blotting, while LldP-His₆ was not detected (FIG. 5B), perhaps due to low levels of expression combined with poor extraction from membranes. Expression of ldhA in Synechococcus resulted in increased levels of intracellular lactate.

The resulting derivative of S. elongatus 7942 was tested along with various controls for the production of D-lactic acid. Lactate production was determined in the supernatant of the culture, which was spun down for 5 min at 21,130×g, using an LD-lactic acid detection kit from R-Biopharm (Marshall, Mich.) adapted for a 96-well plate reader (Victor 3V; Perkin-Elmer) by using 1/10 of the amounts of the reagents recommended by the manufacturer in each assay mixture.

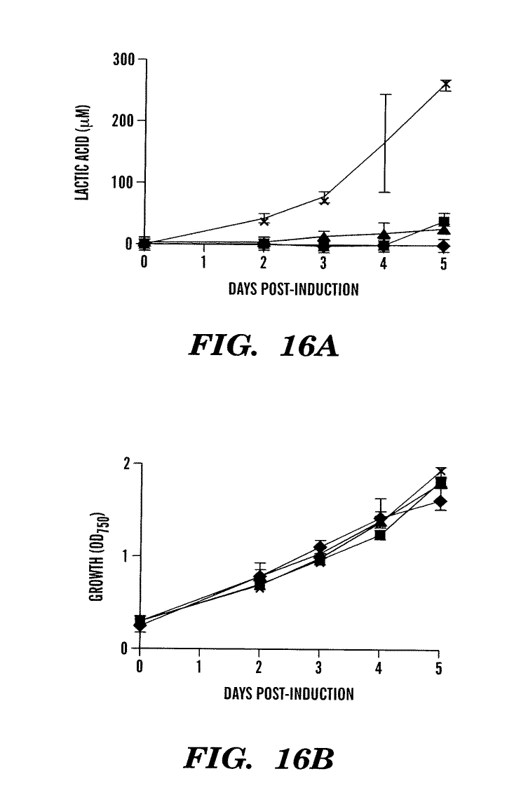

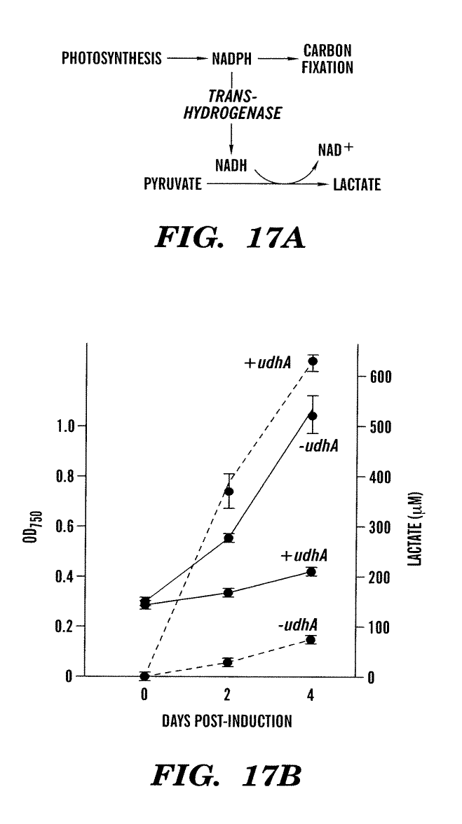

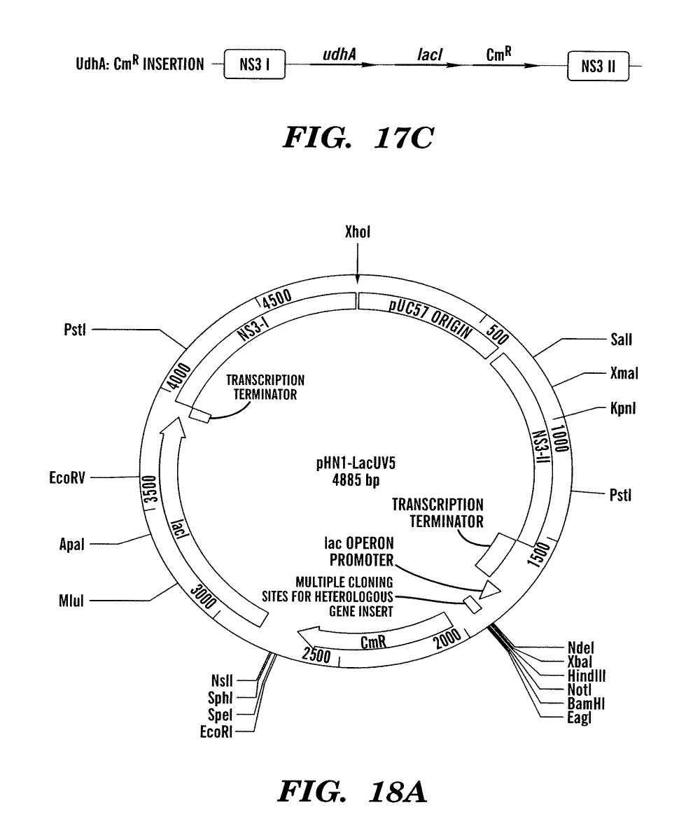

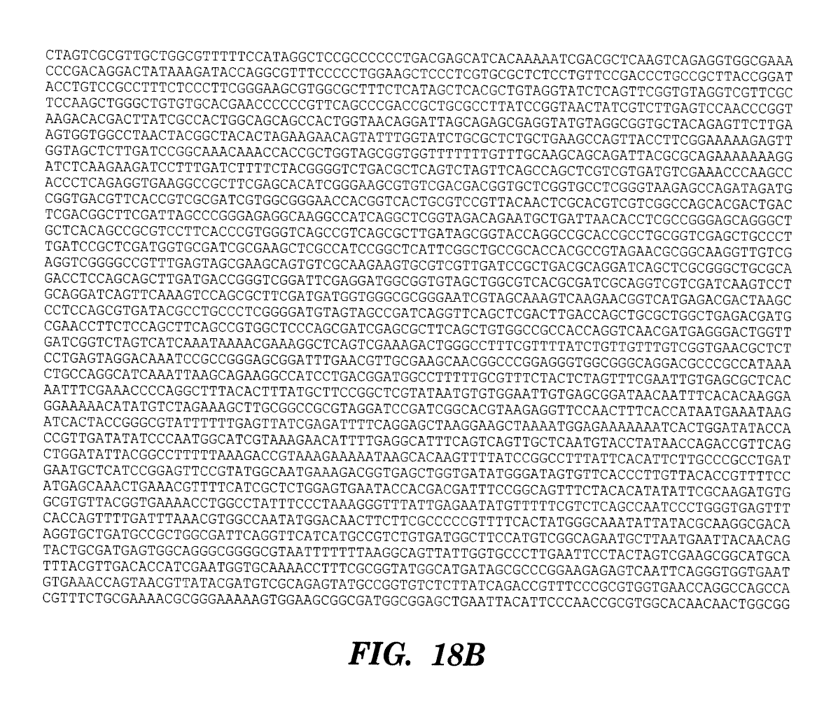

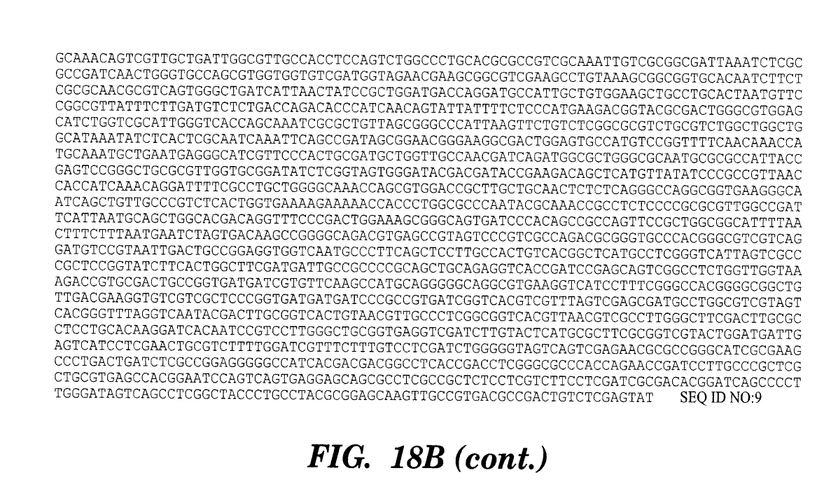

Typical results are shown in FIGS. 17A-17B. Analogous to Synechococcus cells engineered to secrete glucose and fructose in Example 1, Synechococcus cells expressing the E. coli ldhA and lldP genes from IPTG-inducible promoters secreted relatively high levels of lactate into the medium, while cells expressing only ldhA or lldP produced ˜4-fold-lower levels (FIG. 16A), and there was no dramatic difference in the growth rates of these four strains (FIG. 16B). The rate of accumulation of extracellular lactate was much lower than the rate of accumulation of hexose sugars, but it increased steadily for at least 9 days. These observations indicate that the net intracellular rate of lactate production is lower than the net intracellular rate of glucose and fructose production but that after secretion lactate cannot be taken up and metabolized.

### Example 5

### Enhanced Production of Lactic Acid by Additional Engineering

The production of lactic acid is enhanced by the expression of certain additional transgenes. For example, expression of a lactate operon from Lactococcus lactis leads to the production of L-lactic acid. This enantiomer of D-lactic acid is also transported outside the cell by the lldP transporter. This lactate operon includes the genes Lactate dehydrogenase, Pyruvate Kinase and Phosphofructokinase.

Photosynthetic microbes (e.g., S. elongatus 7942) produce NADPH as the major carrier of reducing equivalents, but lactate dehydrogenase uses NADH as its reducing substrate (FIG. 17A). As an alternative or additional method for enhancing lactic acid production, the engineered S. elongatus 7942 strain in Example 4 that expressed the ldhA and lldP genes of E. coli was further engineered to also express the udhA gene of E. coli, which encodes a soluble NADPH/NADH transhydrogenase (Boonstra et al, 181 J. Bacteriol. 1030 (1999)). The IPTG-inducible lac promoter was used.

Production of lactic acid from the resulting strain was compared to production of lactic acid from a corresponding strain expressing only the ldhA and lldP genes of E. coli and containing an empty version of the pHN1-LacUV5 plasmid (FIG. 17B). The diagram of the plasmid with udhA gene insertion is shown in FIG. 17C. Upon induction of all transgenes with IPTG, lactate levels in culture supernatant was markedly enhanced and reached 600 micromolar from the ldhA, lldP, udhA strain of S. elongatus 7942 after four days, while levels of lactic acid in culture supernatant of the ldhA, lldP strain of S. elongatus 7942 accumulated to <100 μM (FIG. 17B). In addition, the ldhA, lldP, udhA strain of S. elongatus 7942 grew extremely slowly after induction with IPTG. This might be due to a flux of energy and carbon into lactate, to alterations in the intracellular pH, or to lower levels of NADPH impacting the regulatory protein OpcA and activating the oxidative pentose phosphate pathway (Hagen et al., 276 J. Biol. Chem. 11477 (2001)). More importantly, the reduction in the growth rate of engineered Synechococcus further expressing the udhA transhydrogenase indicates that on a per cell basis lactic acid by the ldhA, lldP, udhA strain was particularly high compared to its parent strain. Moreover, the initial rate of production of lactate in the strain expressing lactate dehydrogenase, the lactate transporter, and NADP/NAD transhydrogenase was about 54 mg/liter/day/OD₇₅₀ unit (FIG. 17B).

Mutations in other genes to reduce or eliminate the following enzymes also enhances lactate production: malate dehydrogenase, pyruvate dehydrogenase complex, phosphoenolpyruvate carboxylase, phosphoenolpyruvate synthase.

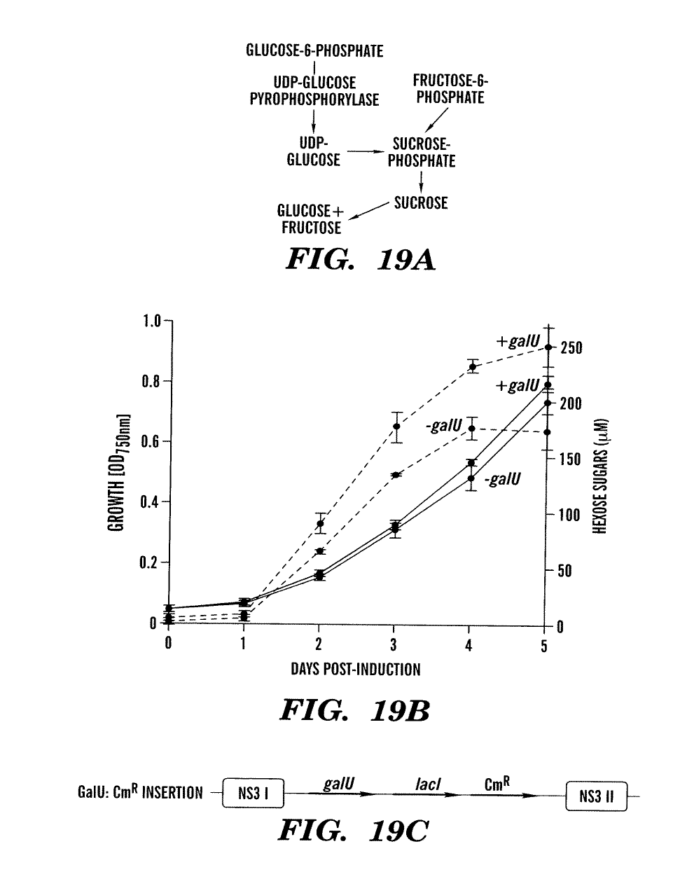

Plasmid and vector construction: To express additional genes for pathway optimization, the udhA gene was introduced into a distinct plasmid integrating vector, termed pHN1-LacUV5, which was designed to integrate at a distinct NS 3 in the S. elongates 7942 genome (FIGS. 19A-19B). The vector expresses E. coli lad and encodes chloramphenicol resistance, mediates integration into the remnant of a cryptic prophage in the Synechococcus genome, and contains a lac operon promoter followed by a multiple-cloning site. To select for homologous recombination and integration of heterologous DNA into the genome and for culture maintenance of engineered strains, kanamycin (25 μg/ml or 2.5 μg/ml if in combination with other drugs), spectinomycin (25 μg/ml or 2 μg/ml if in combination with other drugs) and chloramphenicol (12.5 μg/ml or 2.5 μg/ml if in combination with other drugs) was added to the medium.

### Example 6

### Enhanced Production of Hexose Sugars by Additional Metabolic Engineering

To further demonstrate that secretion of hydrophilic products of interest from Synechococcus could be further enhanced by increasing the levels of intracellular precursors, heterologous enzymes were expressed to improve the production of intracellular sucrose. Without wishing to be bound by theory, the rationale was as follows. Sucrose is normally synthesized from fructose-l-phosphate and UDP-glucose, which are condensed to form sucrose phosphate that is then dephosphorylated to generate sucrose (FIG. 19A). UDP-glucose is synthesized by UDP-glucose phosphorylase, encoded by the E. coli galU gene. Marolda et al., 22 Mol. Microbiol 827 (1996). Integration of the galU gene, encoding UDP-glucose phosphorylase, expressed from an IPTG-regulated promoter at a distinct Synechococcus NS 3 using the pHN1-LacUV5 vector further increased hexose sugar production by more than 30% in cells expressing invA and glf (FIG. 19B). FIG. 19C shows the diagram of the plasmid of galU insertion in Synechococcus.

The market value of commodity organic compounds, such as sugars, lactic acid and amino acids is generally higher than the actual market value of fuels on a per-photon basis. Therefore, engineering photosynthetic microbes to produce and secrete hydrophilic or charged molecules is economically desirable. In addition, biofuels are usually produced by fermentation of sugars. Engineering photosynthetic microbes to produce and secret hydrophilic compounds, such as sugars, contemplates a cost-effective method for biofuel production.

### Example 7

### Expression of a Sucrose/H⁺ Symport Protein in Cyanobacteria

In some embodiments of the present invention use proton symport proteins as transporters for the secretion of metabolites from cyanobacteria. Without wishing to be bound by theory, this is because cyanobacteria tend to alkalinize their growth medium so that when a metabolite is co-transported out of the cell with an H⁺ ion, such transport is essentially irreversible.

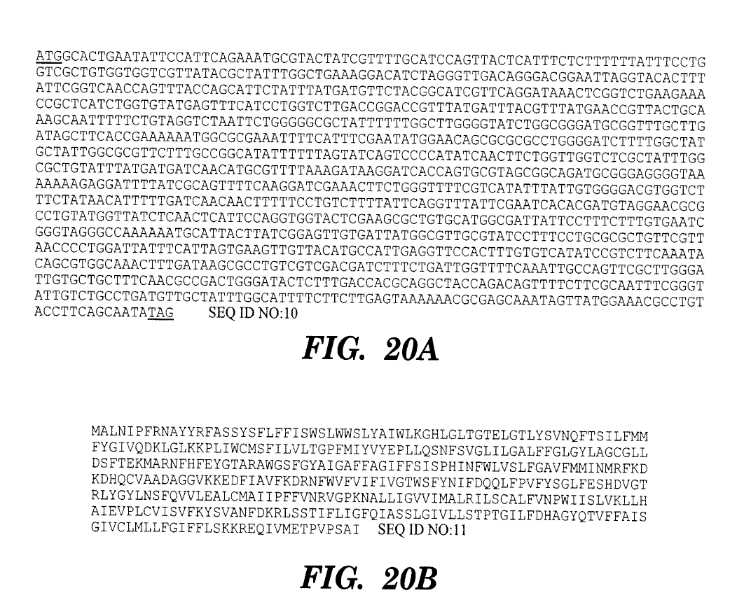

To further illustrate this principle, a cyanobacterium such as Synechococcus elongatus PCC 7942 or Synechocystis species PCC 6803 is engineered to express the sucrose/H⁺ symporter CscB. The CscB protein is similar to LacY permease for lactose (Vadyvaloo et al., 358 J. Mol. Bio. 1051 (2006)); this protein is localized to the plasma membrane, has about twelve transmembrane α-helices, and key amino acids involved in cation co-transport are conserved between the LacY permease and the CscB protein. The nuclei acid and amino acid coding sequences for the CscB protein of E. coli are given in FIGS. 20A and 20B, respectively.

The CscB-encoding sequence is placed in an expression vector for S. elongatus PCC7942 in essentially the same manner as described above for the glf or lldP coding sequences as described above. The promoter used is an inducible promoter such as a derivative of the E. coli lac promoter as described above, or a NaCl-inducible promoter. Alternatively, a constitutive promoter can be used. The resulting CscB expression vector is introduced into S. elongatus PCC7942 by standard transformation procedures known to one of skill in the art. The resulting S. elongatus PCC7942 strain expresses a functional CscB protein.

Evidence for functionality of the CscB protein is demonstrated, for example, as follows. Cyanobacteria expressing the CscB protein can grow very slowly in the absence of light when sucrose is present and the pH is buffered to about pH 7. Concomitant expression of invertase is optionally used to demonstrate this point. Alternatively, when the engineered cyanobacteria are induced to generate intracellular sucrose with NaCl, sucrose is found in the extracellular medium at levels significantly higher than the levels of sucrose in the medium of NaCl-induced, non-engineered S. elongatus PCC7942. For example, when a culture of CscB-expressing S. elongatus PCC7942 at an OD of about 0.05 are induced with 200 mM NaCl, levels of at least about 50 μM sucrose accumulate in the extracellular medium within 2 days. In comparable cultures of non-engineered S. elongatus, the amount of sucrose in the extracellular medium is less than one-half of the levels found in the cultures of the CscB-expressing recombinant S. elongatus.

Production of sucrose may be increased further by expression of enzymes involved in sucrose synthesis, such as sucrose phosphate synthase plus sucrose phosphate phosphatase, and/or such as UDP-glucose phosphorylase as described herein.

### Example 8

### Engineered Synthesis and Secretion of Amino Acids from a Photosynthetic Microbe

Glutamate is a commercially important amino acid, being used as a food additive in the form monosodium glutamate (MSG). To further illustrate aspects of the invention, a cyanobacterium such as Synechococcus elongatus PCC7942 or Synechocystis sp. PCC6803 is engineered to synthesize and secrete glutamate.

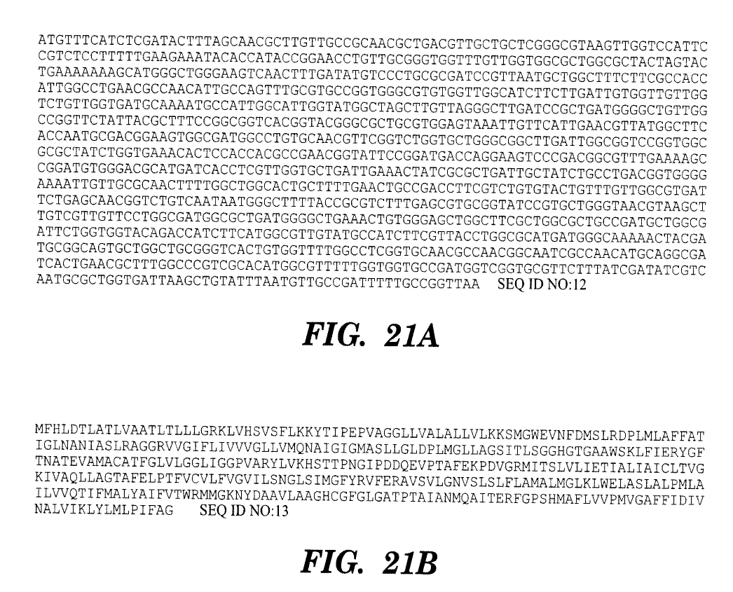

The glutamate transporter GltS promotes secretion of glutamate. GltS co-transports glutamate and Na⁺ ions. Sodium and proton gradients are coupled by a Na⁺/H⁺ antiporter, so that systems involving co-transport with Na⁺ are indirectly coupled to the proton gradient. Thus, metabolites produced by alkalinizing photosynthetic bacteria that are co-transported with Na⁺ will accumulate outside of the cell, just as metabolites co-transported with H⁺ will accumulate outside of the cell. The nuclei acid and amino acid coding sequences for the GltS protein of E. coli are given in FIGS. 21A and 21B.

The GltS-encoding sequence is placed in an expression vector for S. elongatus PCC7942 in essentially the same manner as described above for the glf or lldP coding sequences as described above. The promoter used is an inducible promoter such as a derivative of the E. coli lac promoter as described above, or a NaCl-inducible promoter. Alternatively, a constitutive promoter can be used. The resulting GltS expression vector is introduced into S. elongatus PCC7942 by standard transformation procedures known to one of skill in the art. The resulting S. elongatus PCC7942 strain expresses a functional GltS protein.

Evidence for functionality of the GltS protein is demonstrated, for example, as follows. When the recombinant cyanobacteria are induced to express GltS, glutamate accumulates in the extracellular medium. In comparable cultures of non-engineered S. elongatus, the amount of glutamate in the extracellular medium is less that one half of the levels found in the cultures of the recombinant, GltS-expressing S. elongatus.

1. A recombinant photosynthetic bacterium comprising at least one heterologous enzyme and at least one heterologous transporter, wherein said transporter mediates the secretion of a product whose synthesis is enhanced by said enzyme.

2. The recombinant photosynthetic bacterium of claim 1, wherein the transporter is a member of the major facilitator superfamily.

3. The recombinant photosynthetic bacterium of claim 1, wherein the heterologous transporter encoded by a gene selected from the group consisting of: the Glut family of glucose transport proteins in humans and other mammals; the HXT family of glucose transporters in yeast; lacY; lldP lactate transporter of E. coli or another bacterium that performs anaerobic metabolism; a mammalian citric acid transport protein; the glpT glycerol phosphate transporter; and GLF of Z. mobilis.

4. The recombinant photosynthetic bacterium of claim 1, wherein the enzyme is an invertase or a lactate dehydrogenase.

5. The recombinant photosynthetic bacterium of claim 1, further comprising a reduction-of-function mutation in an endogenous hexokinase gene, an endogenous glucokinase gene, or both, malate dehydrogenase, pyruvate dehydrogenase complex, phosphoenolpyruvate carboxylase, phosphoenolpyruvate synthase, or a combination thereof.

6. The recombinant photosynthetic bacterium of any of claim 1, wherein the product is a chiral compound or a hydrophilic product.

7. The recombinant photosynthetic bacterium of claim 6, wherein the chiral compound or the hydrophilic product is a hexose sugar or lactic acid.

8.-12. (canceled)

13. The recombinant photosynthetic bacterium of claim 4, wherein the enzyme is expressed from an inducible promoter.

14. The recombinant photosynthetic bacterium of claim 1, wherein the photosynthetic bacterium is a cyanobacterium.

15. (canceled)

16. The recombinant photosynthetic bacterium of claim 1, wherein the photosynthetic bacterium is additionally engineered to express a soluble NAD(P) transhydrogenase, a UDP-glucose phosphorylase or both.

17. (canceled)

18. A recombinant photosynthetic bacterium comprising at least one heterologous transporter, wherein said transporter mediates the symport or antiport of a product and an inorganic ion.

19. The recombinant photosynthetic bacterium of claim 18, wherein said inorganic ion is selected from the group consisting of a proton, a sodium ion, a potassium ion, a chloride ion, and a hydroxide ion.

20. The recombinant photosynthetic bacterium of claim 18, wherein said transporter is selected from the group consisting of a lactic acid/proton symporter, a sucrose proton symporter, a lactose/proton symporter, a dicarboxylic acid/proton symporter, an amino acid/proton symporter, a citrate/proton symporter, an amino acid/sodium ion symporter, and an amino acid/hydroxide ion antiporter.

21. The recombinant photosynthetic bacterium of claim 18, wherein the heterologous transporter encoded by a gene selected from the group consisting of: the sucrose transporter CscB; the lactose permease of E. coli encoded by the lacY gene; the mammalian H+/peptide transporter PepT1H; the glutamate transporter GltS and the lactate transporter lldP.

22. The recombinant photosynthetic bacterium of claim 18, wherein the product is a chiral product, an amino acid or a sugar.

23. (canceled)

24. The recombinant photosynthetic bacterium of claim 22, wherein the amino acid is glutamate.

25. (canceled)

26. The recombinant photosynthetic bacterium of claim 18, further comprising at least one more heterologous gene.

27. The photosynthetic bacterium of claim 26, wherein the heterologous gene encodes an enzyme or a transporter.

28. The photosynthetic bacterium of claim 27, wherein the enzyme is ldhA.

29. A method for producing a product, said method comprising culturing a recombinant photosynthetic bacterium in the presence of light and carbon dioxide, and obtaining the product from the supernatant of the cultured bacterium, wherein the recombinant photosynthetic bacterium comprises at least one heterologous enzyme and at least one heterologous transporter, wherein said transporter mediates the secretion of a product whose synthesis is enhanced by said enzyme; or wherein the recombinant photosynthetic bacterium comprises at least one heterologous transporter, wherein said transporter mediates the symport or antiport of a product and an inorganic ion.

30. (canceled)
